(19) United States
(12) Patent Application Publication
(10) Pub. No.: US 2024/0335811 A1
(43) Pub. Date: Oct. 10, 2024

(54) Microcapsules and Methods for Preparing Microcapsules

(71) Applicant: CPL AROMAS LIMITED, Brixworth, Northampton (GB)

(72) Inventor: Martin Zaki, Brixworth, Northampton (GB)

(21) Appl. No.: 18/579,291

(22) PCT Filed: Jul. 14, 2022
(86) PCT No.: PCT/EP2022/069777

§ 371 Date: Jan. 12, 2024

(30) Foreign Application Priority Data
Jul. 14, 2021 (GB) 2110132.4

(51) Int. Cl. B01J 13/10; A61K 8/11; B01J 13/16; C11D 3/00; C11D 3/22; C11D 3/37; C11D 3/50; C11D 17/00

(52) U.S. Cl. CPC B01J 13/10; A61K 8/11; B01J 13/16; C11D 3/0015; C11D 3/222; C11D 3/3726; C11D 3/505; C11D 17/0039

(57) ABSTRACT

The invention provides a method for producing a microcapsule. The method involves inducing encapsulation of an organic phase by a complex coacervate and polymerising a polyisocyanate at an interface of the organic phase, and adding a crosslinking agent to form crosslinks. The invention also provides a microcapsule obtained by the method, a composition having a plurality of microcapsules and a consumer product. The microcapsules may be used to encapsule a cargo. Also provided is a composition for preparing a microcapsule. The composition includes a polycation and a polyanion in an aqueous phase, a polyisocyanate in an organic phase, and a crosslinking agent.

### RELATED APPLICATION

The present case claims priority to, and the benefit of, GB 2110132.4 filed on 14 Jul. 2021 (14 Jul. 2021), the contents of which are hereby incorporated by reference in their entirety.

### TECHNICAL FIELD

This invention relates to microcapsules for holding an encapsulant, such as a fragrance, and compositions containing the microcapsule. The invention also relates to methods for preparing microcapsules and their use in encapsulation.

### BACKGROUND

Microcapsules are used to isolate, stabilise and deliver active agents into several diverse consumer products. Examples include the use of microcapsules for protecting sensitive vitamins to oxidation, or to deliver the active at a targeted time or zone.

Fragrances are used to give an appealing odour in consumer products. In particular, they are associated with “cleanliness” or “freshness” within fabric care applications. Although fragrances can be directly added to fabric within a wash cycle, the transfer is usually minimal and the more volatile materials, such as those delivering the top notes, fade quickly. Encapsulating fragrances not only overcomes the technological hurdle of delivering a suitable olfactive performance and stability, it also reduces the amount of fragrance required to achieve the same performance. The long-lasting performance that is delivered by encapsulation allows the consumer to smell the pleasant fragrance that the consumer will associate with cleanliness days after a wash.

Existing technologies for microencapsulation are based on the use of synthetic or natural polymers for the formation of the microcapsule shell. Microcapsules made from synthetic polymers, such as melamine formaldehyde or polyacrylate, are effective in maintaining the fragrance. However, such polymers do not address the growing demand from both consumers and regulators for environmentally benign materials. Natural polymers used in the preparation of technologies such as complex coacervates or gelation offer a more sustainable alternative. These technologies offer better adhesion and physical properties that flexibly change depending on moisture content, but these materials offer poor protection to small volatile materials. The diffusion rate of volatile materials through these shells is quick and leads to hasty leakage. A large share of the commercial fragrance microcapsule market is in fabric care such as fabric conditioners and liquid laundry compositions. These surfactant rich environments increase the diffusion rate drastically and the market has seen a growth in reducing water content and increasing concentration of surfactants which leads to an ever-greater requirement for stable microcapsules.

Following consumer concern for plastic pollution and more specifically for microplastic pollution, there is an interest in alternative options to existing synthetic microcapsules that are readily degradable in the natural environment.

US 2015/250689 produces multi-layered microcapsules by initially coating the core with a Gelatine-Gum Arabic complex coacervate, and then inducing interfacial polymerisation with the use of a reactant with the monomer present in the core. US 2019/0240124 discloses a method to produce multi-layered microcapsules without the addition of a reactant. US 2005/0112152, US 2020/0281826, and WO 2019/096817, all describe various methods of coating an existing microcapsule with a cationic polymer. WO 2021/018947 discloses multi-layered microcapsules having a composite shell with a first material that is a coacervate, and a polymeric second material where the weight ratio between the first and second material is between 50:50 and 99.9:0.1.

Although some of these multi-layered microcapsules are suitable to deliver a satisfactory olfactive performance, they are not suitable for environmental degradability.

As of recent years, there has been development of biodegradable microcapsules. CN 106614564 describes a matrix microcapsule compromising ethyl cellulose and modified starch that diffuses over a period of 72 hours.

US 2011/0111020 creates an initial complex coacervate shell followed by additional coatings of a natural polymer surrounding several initial microcapsules. Although the polymers used are biodegradable, the materials and methods used produce a highly porous shell that would be unsuitable for volatile materials, such as fragrances.

US 2020/0164332 encapsulates a water in oil emulsion by re-emulsifying the first emulsion into water and photo-initiating polymerisation. Although the microcapsules were found to be biodegradable, it is not clear whether the capsule material itself is biodegradable. In fact, there is a direct correlation between the increase in the encapsulated active material and the increase in biodegradability, suggesting that the inner phase may be the only material contributing to the biodegradability.

WO 2020/131866 describes interfacial polymerised microcapsules using a polysaccharide and isocyanate fabricating a polyurethane shell, and WO 2020/131890 also describes interfacial polymerised microcapsules using chitosan, isocyanate and a polyphenol. Contrary to the synthetic polymer microcapsules, the biodegradable capsules lack the quality required to suit consumer needs.

### SUMMARY OF THE INVENTION

At its most general, the present invention relates to a microcapsule having a shell comprising a complex coacervate and a polymer, which shell is reinforced by crosslinking the shell components. The microcapsule can hold an encapsulant for later release.

The microcapsules of the present invention are useful for encapsulating a volatile substance such as a fragrance or perfume. The microcapsules have good stability and control, such as delay, diffusion of the encapsulant through the microcapsule shell. The microcapsules also have good release performance by allowing efficient deposition of the encapsulant onto a substrate, such as clothing fabric. The microcapsules are degradable, such as biodegradable, and do so over a time period that is desirable within the fragrance, laundry and personal care industries, such as over a period of 28 days. For example, the microcapsules degrade by at least 60% within a period of 28 days as measured under standard conditions, such as the conditions set out in the OCED TG 301F test. ECHA has identified that a minimum of 60% biodegradability in 60 days is required to combat the microplastic endemic.

In a first aspect, the invention provides a method for producing a microcapsule, comprising the steps of:
(i) providing a complex coacervate of a polycation with a polyanion in an aqueous phase;
(ii) dispersing an organic phase comprising a polyisocyanate in the aqueous phase;
(iii) inducing encapsulation of the organic phase by the complex coacervate and polymerising the polyisocyanate at the interface of the organic phase; and
(iv) adding a crosslinking agent to the dispersion of step (iii) in an amount of up to 0.03% by weight of the dispersion and permitting the crosslinking agent to form crosslinks.

The polymerisation of the polyisocyanate proceeds together with encapsulation of the organic phase by the complex coacervate during step (iii), to form a microcapsule shell comprising a complex coacervate and a polymer, such as a polyurethane. This provides a microcapsule having good stability and good performance, which is further reinforced by the crosslinking performed in step (iv). A crosslinking agent is permitted to form crosslinks between the components of the shell, such as the polycation and the polyanion in the coacervate and the polymerisation product of the polyisocyanate.

The amount of the crosslinking agent is used at a level to provide a balance between the stability of the microcapsules, and biodegradability. The amount of the crosslinking agent added may be 0.001% to 0.03% by weight of the dispersion. In this way, the microcapsules may be degradable, such as biodegradable.

The amount of the crosslinking agent added may be expressed relative to the amount of coacervate, additionally or alternatively to the amount relative to the dispersion. For example, the amount of the crosslinking agent added may be up to 10 parts by weight relative to 1,000 parts by weight of the complex coacervate provided in step (i), preferably up to 5 parts by weight relative to 1,000 parts by weight of the complex coacervate. In this way, the microcapsules may have good stability and performance whilst also being degradable, such as biodegradable, such as inherently biodegradable.

Step (iv) may comprise a stage of permitting the crosslinking agent to form crosslinks at a temperature of 30° C. or more, such as 30° C. to 50° C. Step (iv) may comprise heating the dispersion to a temperature of 30° C. or more in the presence of the crosslinking agent. This accelerates the crosslinking process and improves the crosslinking yield. Step (iv) may comprise two or more stages that are carried out at different temperatures. Preferably, step (iv) comprises a stage of adding a crosslinking agent at ambient temperature and permitting the crosslinking agent to form crosslinks, followed by adjusting the temperature to 30° C. or more.

The polycation may be selected from a polysaccharide, a plant protein and a milk protein. Preferably, the polycation is a polysaccharide, such as chitosan. The polyanion may be selected from cellulose, starch, modified starch, agar, alginate, xanthan gum, plant gum, casein, zein, agarose, polyvinyl alcohol, isosorbide, derivatives thereof and salts thereof. Preferably, the polyanion is a plant gum, such as acacia gum. The use of a coacervate derived from these components provides a microcapsule that is biodegradable whilst providing good stability and good release performance. Moreover, these polymers are naturally derived and are therefore more sustainable than the use of synthetic polymers.

In some preferred embodiments, the polycation is chitosan and the polyanion is acacia gum. In other preferred embodiments, the polycation is chitosan and the polyanion is carboxymethyl cellulose.

The polycation and/or the polyanion may be derivable from plant or fungal sources, and thus do not require the use of animal products.

The coacervate in the microcapsule shell is provided for adhesion of the microcapsule onto substrates such as fabric, whilst the polymer in the microcapsule shell improves chemical stability,

The crosslinking agent is not particularly limited, and is typically an agent that is capable of forming covalent links between the polymeric components of the microcapsule, such as the polycation, the polyanion, and/or the polymerisation product of the polyisocyanate. The crosslinking agent may be selected from glutaraldehyde, formaldehyde, acetaldehyde, glyoxal, epichlorohydrin, transglutaminase, tannic acid, sodium tripolyphosphate, boric acid, genipin, N-(3-dimethylaminopropyl)-N′-ethylcarbodiimide hydrochloride, bis(triethoxysilylpropyl) tetrasulfide, vinyltriethoxysilane, vinyltrimethoxysilane, dimethyl suberimidate, 1,2-cyclopentanedione and 1,3-cyclopentanedione. Preferably, the crosslinking agent is selected from glutaraldehyde, formaldehyde, acetaldehyde and glyoxal. Most preferably, the crosslinking agent is glutaraldehyde. The crosslinking agent reacts with the components of the complex coacervate and/or the polymerisation product of the polyisocyanate to strengthen the microcapsule. This contributes to the stability and performance of the microcapsule, and when the crosslinking agent is used at the specified amount in the dispersion, it does so without compromising biodegradability.

Step (i) may comprise forming the complex coacervate from the polycation and the polyanion. In other embodiments, the complex coacervate may be provided pre-formed in the aqueous phase.

In step (ii) the dispersing of the organic phase may comprise forming droplets of the organic phase in the aqueous phase. In these embodiments, the droplets of the organic phase may be encapsulated by the complex coacervate in step (iii), and polymerisation of the polyisocyanate may be at the interface of the droplets. Typically, a droplet is substantially spherical. The dimensions of the droplets may be substantially homogeneous across a sample population.

The polyisocyanate may comprise an aromatic group. The use of a polyisocyanate comprising an aromatic group provides increased steric hinderance which strengthens the microcapsule shell and improves the microcapsule stability. This can be seen from improved stability of the example microcapsule Samples 1, 2, 4 and 5 below, which are prepared using a polyisocyanate comprising aromatic groups, when compared to Sample 3 below was prepared using a more aliphatic polyisocyanate.

The polyisocyanate may be a diisocyanate. The polyisocyanate may comprise one or more of xylylene diisocyanate (XDI), hydrogenated xylylene diisocyanate (HXDI), toluene diisocyanate, hexamethylene diisocyanate (HDI), pentamethylene diisocyanate, methylene diphenyl diisocyanate, and adducts thereof, and oligomers thereof. In some embodiments, the adduct is a trimethylol-propane adduct. In some embodiments, the oligomer is a selected from a dimer, a trimer, an uretidione, an isocyanurate, an iminooxadiazinedione, an allophanate and a biuret. One or more of these polyisocyanates may be used in the step (ii).

Step (iii) may include adjusting the pH of the mixture. In some embodiments, the pH of the mixture is adjusted by addition of a reagent to increase or decrease the pH. In some embodiments, a base is added to increase the pH of the mixture. Step (iii) may be performed at a temperature from 15° C. to 30° C., such as at ambient temperature. These pH and/or temperature conditions promote encapsulation of the organic phase by the complex coacervate, and also encourages interfacial polymerisation to occur simultaneously with the encapsulation.

In some embodiments, steps (i) and (ii) are performed sequentially. Step (i) may be performed followed by step (ii), or step (ii) may be performed followed by step (i). For example, the organic phase may be dispersed in the aqueous phase first, followed by introducing a complex coacervate into the aqueous phase. In other embodiments, the steps (i) and (ii) may be performed simultaneously.

The organic phase may comprise one or more components for encapsulation, for example one or more of a fragrance, a cosmetic active, a malodour counteractive, an antimicrobial agent and an insecticide. These types of cargo may comprise volatile substances, and encapsulation by the method of the present invention limits release of the substances from the organic phase.

The steps (i), (ii), (iii) and (iv) may each comprise mixing. Mixing during step (iv) in particular helps to prevent agglomeration of the microcapsules.

In a second aspect, the invention provides a microcapsule that is obtainable by or obtained by the method of the first aspect.

The microcapsule may have a largest dimension, such as a diameter, of 0.1 μm to 1,000 μm. The largest dimension may be in the range 5 μm to 150 μm, such as 40 μm to 60 μm. Microcapsules of this size can be effectively deposited onto a substrate, such as fabric by being trapped in the fabric. If the microcapsules are too small, agglomeration may occur between microcapsules. If the microcapsules are too small and the microcapsule shells are also thin, this may lead to reduced stability.

The microcapsule may have a shell thickness that is from 0.01 μm to 200 μm, such as from 1 μm to 10 μm. A thicker microcapsule shell has the advantage of increased stability. The thickness of the microcapsule shell may be altered by changes to the materials used in the shell, and their absolute and relative amounts.

In a third aspect, the invention provides a composition comprising the microcapsules of the second aspect of the invention. The composition may be an aqueous composition. The microcapsules may be provided as a suspension in the composition.

The composition may further comprise a surfactant, preservative, pH stabiliser, thickening agent, opacifier, sequestrant, propylene glycol, salt, or a combination thereof.

In a fourth aspect, the invention provides a consumer product comprising the composition of the third aspect of the invention. The consumer product may be
a personal care product selected from an antiperspirant cream, a antiperspirant spray, a deodorant, a roll on, a deodorant stick, a liquid shampoo, a liquid shower gel, a liquid hair conditioner, a solid shampoo, a solid shower gel, a solid hair conditioner, a body wash, a bath fragrance, a bath bomb, a bath salt, a soap, a soap bar, a hand wash, a hand sanitiser, and hand gel, a body cream, a hand moisturiser, a face moisturiser, a hair gel, a hair wax, a hair mousse, a hair dye, a hair bleach, a shaving cream, a shaving paste, a shaving powder, a face mask, a depilatory cream, and a fake tan;
a fabric care product selected from a scent booster, a liquid laundry detergent, a liquid fabric conditioner, a detergent powder, a fabric conditioner powder, a laundry detergent sheet, a fabric detergent sheet, a fabric refresher, a laundry prewash, a laundry aid, an ironing water, and a leather care product;
a home care product selected from a furniture polish, a floor cleaner, a window cleaner, a surface cleaner, an all-purpose cleaner, a carpet shampoo, a bleach, a toilet cleaner, a toilet block, a urinal cake, a de-scaler, a disinfectant, a washing up liquid, a solid washing up soap, a dishwasher tablet, a dishwasher powder, and a dishwasher sheet;
a fragrance product selected from a diffuser, a candle, an air refresher, a reed diffuser, a incense stick, a car wash, a fine fragrance product, an Eau De Toillette, an Eau de Parfum, a cologne, a solid perfume, a perfume gel, and a scratch and sniff card; and
a textile product selected from a padded textile, and a clothing item.

In a fifth aspect, the invention provides a use of the microcapsule as described herein to hold a cargo selected from one or more of a fragrance, a cosmetic active, a malodour counteractive, and an antimicrobial agent or an insecticide.

In an additional or alternative aspect of the invention, there is provided a composition for preparing a microcapsule, the composition comprising:
an aqueous phase comprising a polycation and a polyanion;
an organic phase comprising a polyisocyanate; and
a crosslinking agent,
wherein the crosslinking agent is present at an amount of up to 0.03% by weight of composition.

The amount of the crosslinking agent present may alternatively or additionally be expressed relative to the total amount of polycation and polyanion. For example, the amount of the crosslinking agent added may be up to 10 parts by weight relative to 1,000 parts by weight of the total weight of polycation and polyanion, such as from 0.1 to 10 parts by weight relative to 1,000 parts by total weight of the polycation and the polyanion. Preferably the amount of the crosslinking agent is up to 5 parts by weight relative to 1,000 parts by total weight of the polycation and polyanion, more preferably up to 4 parts by weight.

The aqueous phase may comprise a complex coacervate formed between the polycation and the polyanion.

The preferred features of the first and second aspects apply equally to the sixth aspect.

These and other aspects and embodiments of the invention are described in further detail below.

### SUMMARY OF THE FIGURES

The present invention is described with reference to the figures listed below.

FIG. 1 shows the stability and performance results at room temperature for microcapsule samples 1 to 5, dosed into Fabric Conditioner. The rubbed performances at 2, 4, 8 and 12 weeks are shown.

FIG. 2 shows the stability and performance results at 40° C. for microcapsule samples 1 to 5, dosed into Fabric Conditioner. The rubbed performances at 2, 4, 8 and 12 weeks are shown.

FIG. 3 shows the stability performance results at room temperature for microcapsule samples 1 to 5, dosed into Liquid Laundry. The rubbed performances at 2, 4, 8 and 12 weeks are shown.

FIG. 4 shows the stability and performance results at 40° C. for microcapsule samples 1 to 5, dosed into Liquid Laundry. The rubbed performances at 2, 4, 8 and 12 weeks are shown.

FIG. 5 shows the stability performance results at room temperature for microcapsule samples 1, 5 and 6, dosed into Fabric Conditioner. The rubbed performances at 2, 4, 8 and 12 weeks are shown.

FIG. 6 shows the stability performance results at 40° C. for microcapsule samples 1, 5 and 6, dosed into Fabric Conditioner. The rubbed performances at 2, 4, 8 and 12 weeks are shown.

FIG. 7 shows the stability performance results at room temperature for microcapsule samples 1, 5 and 6, dosed into Liquid Laundry. The rubbed performances at 2, 4, 8 and 12 weeks are shown.

FIG. 8 shows the stability performance results at 40° C. for microcapsule samples 1, 5 and 6, dosed into Liquid Laundry. The rubbed performances at 2, 4, 8 and 12 weeks are shown.

FIG. 9 shows the stability performance results at room temperature for comparative microcapsule samples dosed into Fabric Conditioner. The rubbed performances at up to 12 weeks are shown.

FIG. 10 shows the stability performance results at 40° C. for comparative microcapsule samples dosed into Fabric Conditioner. The rubbed performances at up to 12 weeks are shown.

FIG. 11 shows the stability performance results at room temperature for comparative microcapsule samples dosed into Liquid Laundry. The rubbed performances at up to 12 weeks are shown.

FIG. 12 shows the stability performance results at 40° C. for comparative microcapsule samples dosed into Liquid Laundry. The rubbed performances at up to 12 weeks are shown.

FIG. 13 shows Laundrometer performance results of microcapsule samples dosed into Fabric Conditioner, smelt before and after rubbing.

FIG. 14 shows biodegradability as % ThOD over 60 days for samples 1, 3, 4 and 5. Pure oil and reference material comparison are also shown on each plot. The solid horizontal line indicates 10% biodegradability, and vertical line marks end of lag period. The 10-day window is shaded in grey. The horizontal line indicates 90% biodegradability.

FIG. 15 shows biodegradability as % ThOD for the reference only treatment (seed subtracted) and reference+oil toxicity test (seed+oil) treatment subtracted to show effect on reference material.

FIG. 16 shows stability performance results at room temperature for microcapsule samples 1, 5, 6 and comparative sample 7 dosed into Fabric Conditioner. The rubbed performances at 2, 4, 8 and 12 weeks are shown.

FIG. 17 shows stability performance results at 40° C. for microcapsule samples 1, 5, 6 and comparative sample 7, dosed into Fabric Conditioner. The rubbed performances at 2, 4, 8 and 12 weeks are shown.

FIG. 18 shows stability performance results at room temperature for microcapsule samples 1, 5, 6 and comparative sample 7, dosed into Liquid Laundry. The rubbed performances at 2, 4, 8 and 12 weeks are shown.

FIG. 19 shows stability performance results at 40° C. for microcapsule samples 1, 5, 6 and comparative sample 7, dosed into Liquid Laundry. The rubbed performances at 2, 4, 8 and 12 weeks are shown.

FIG. 20 shows biodegradability as % ThOD over 33 days for microcapsule samples B, C and D (dark grey). The Pure oil (black) and reference material (light grey) are also shown on each plot. The horizontal line indicates 10% biodegradability, and the vertical line marks end of lag period. The 10-day window is shaded in light grey.

FIG. 21 shows biodegradability as % ThOD achieved at 28 days for microcapsule samples 1, 5, B, C and D (as shown in Table 17) plotted against coacervate:glutaraldehyde ratio. The trendline is logarithmic (R²=0.9212).

FIG. 22 shows biodegradability as % ThOD achieved at 28 days for microcapsule samples 1, 5, B and D (as shown in Table 17) plotted against log (coacervate:glutaraldehyde ratio). The trendline is linear (R²=0.9882)

FIG. 23 shows the FTIR spectrum of microcapsule shells of samples A (light grey), B (dark grey) and C (black) in the range of 4000 to 400 cm-1.

FIG. 24 shows an FTIR spectrum of microcapsule shells of samples A (light grey), B (dark grey) and C (black) in the range of 1850 to 700 cm-1.

FIG. 25 shows a plot of relative area measured in the IR spectra of microcapsule samples A, B and C for the indicated stretches.

FIG. 26 shows washing machine performance results of microcapsule samples dosed into Fabric Conditioner, smelt before and after rubbing.

### DETAILED DESCRIPTION OF THE INVENTION

At its most general, the present invention relates to a microcapsule having a shell comprising a complex coacervate and a polymer, which shell is reinforced by crosslinking the shell components. The microcapsule can hold an encapsulant for later release.

An important parameter for the encapsulation of cargo, such as fragrances or perfume, within a microcapsule is the stability of that microcapsule. Depending on the stability of the microcapsule, the fragrance, which can often contain volatile compounds, may diffuse over time into a base formation in which the microcapsule is suspended. Base formulations can provide a harsh environment for microcapsules. For example, the stability of some microcapsules can be poor in the presence of surfactants.

The performance of microcapsules is another important parameter. If the stability is good, the microcapsule shell protects the encapsulated perfume during storage up until controlled release of the perfume. On the other hand, if the stability is poor then the long-lasting performance that is desirable for microcapsules is no longer possible. The determining factor during performance testing, following stability, is the deposition onto a test substrate. If the deposition is poor, not enough fragrance is deposited onto the substrate to give a suitable strength.

At the same time as ensuring good stability and good performance of microcapsules, it is also desirable for microcapsules to be biodegradable for environmental reasons. It is particularly desirable for the microcapsules to degrade into smaller components that are themselves benign to the environment.

WO 2016/185171 describes microcapsules wherein the microcapsule shell includes a coacervate comprising chitosan, which is crosslinked. The microcapsules do not comprise a polyurethane. The amount of crosslinking agent used for the crosslinking is higher than that in the present invention (>0.2 wt. %). The biodegradability of the microcapsules is not measured. In addition, the coacervates generally offer poor protection for small volatile materials. This can be seen by the stability of Comparative Sample 8 below, where a microcapsule comprising chitosan-based coacervate is tested, which decreases substantially when stored at either room temperature or at 40° C. over a period of 1 to 12 weeks.

US 2015/250689 and US 2019/240124 describe methods for making composite shell microcapsules which include forming an outer shell by coacervation and then forming an inner shell by interfacial polymerisation. A crosslinking agent is used to crosslink the composite shell. Gelatin is used as the polycation in the coacervate. The biodegradability of the microcapsules is not tested or commented on. The amount of crosslinking agent added is up to around 0.04% by weight of the total microcapsule dispersion for glutaraldehyde, and around 0.67% for transglutaminase. As can be seen from the Comparative Sample 9 below, microcapsules that utilise a gelatin-based coacervate have poorer release performance compared to other coacervates, for example those using chitosan.

WO 2021/018947 describes composite microcapsules including a coacervate and a polymeric material together in a microcapsule shell. A microcapsule prepared using a polyisocyanate, gum Arabic and chitosan is disclosed. The microcapsules are subjected to crosslinking using glutaraldehyde at a concentration of 0.04% of the total microcapsule dispersion. In the present invention, a crosslinking agent is added in an amount of up to 0.03% by weight of the dispersion. The biodegradability of the microcapsules is not measured, and it is also not reported that the microcapsules have the stability and performance suitable for encapsulating small volatile compounds.

The present inventors have established that a microcapsule may be prepared from a complex coacervate and a polyisocyanate. The microcapsules are obtainable by encapsulation of the organic phase by the complex coacervate, together with polymerisation of the polyisocyanate around the organic phase. The polymerisation of the polyisocyanate and the encapsulation by the coacervate provides a microcapsule shell around the organic phase, which is reinforced by crosslinking. The microcapsules are surprisingly found to have good stability and good performance over a period of up to 12 weeks, when suspended in a base composition. The microcapsules are also capable of biodegrading.

The combination of two encapsulation technologies, coacervation and interfacial polymerisation, provides a system that meets both stability and performance requirements whilst also being biodegradable. By permitting interfacial polymerisation to occur together with the formation of a coacervate shell, these two technologies are combined together advantageously to form a polymeric network. During interfacial polymerisation, the polyisocyanate reacts with one or more of water, the polycation and the polyanion. In this way, the coacervate and the polymer can be interwoven within the microcapsule shell.

The present inventors have also established that the degree of crosslinking influences the biodegradability of the microcapsules. Relevant factors include the amount of crosslinking agent used and may also include other reaction conditions during crosslinking, such as the reaction temperature and the reaction time.

The invention provides a method for producing a microcapsule, comprising the steps of:
(i) providing a complex coacervate of a polycation with a polyanion in an aqueous phase;
(ii) dispersing an organic phase comprising a polyisocyanate in the aqueous phase;
(iii) inducing encapsulation of the organic phase by the complex coacervate and polymerising the polyisocyanate at the interface of the organic phase; and
(iv) adding a crosslinking agent to the dispersion of step (iii) in an amount of up to 0.03% by weight of the dispersion and permitting the crosslinking agent to form crosslinks.

In alternative embodiments, the amount of crosslinking agent added may be expressed relative to the complex coacervate. Thus, the invention also provides a method for producing a microcapsule, comprising the steps of:
(i) providing a complex coacervate of a polycation with a polyanion in an aqueous phase;
(ii) dispersing an organic phase comprising a polyisocyanate in the aqueous phase;
(iii) inducing encapsulation of the organic phase by the complex coacervate and polymerising the polyisocyanate at the interface of the organic phase; and
(iv) adding a crosslinking agent to the dispersion of step (iii) in an amount of up to 10 parts by weight relative to 1,000 parts by weight of the complex coacervate provided in step (i), and permitting the crosslinking agent to form crosslinks.

### Complex Coacervate

The microcapsule of the present invention comprises a complex coacervate of a polycation with a polyanion.

The complex coacervate is provided in step (i) of the method as a mixture in an aqueous phase. Complex coacervation occurs between oppositely charged polyelectrolytes. The complex coacervate may be provided as droplets in the aqueous phase, for example.

A polycation is a cationic polymer. By “cationic polymer”, it is meant a polymer having a net positive charge. In the complex coacervate, the polycation may be the counterion to the polyanion.

The polycation may be selected from a polysaccharide and a protein.

A polysaccharide is a polymer comprising multiple repeating saccharide units. A polysaccharide may comprise monosaccharide, disaccharide, oligosaccharide units, or a combination thereof. An example of a polysaccharide is chitosan.

A protein is a polymer comprising amino acid monomer units. Examples of a protein include a protein obtainable from an animal, plant or fungus. The polycation may be a naturally occurring protein, such as a protein isolate obtainable from, or obtained from, an animal source, plant source or fungal source. The polycation may be additionally modified following isolation. For example, the polycation may be a hydrolysed form of a naturally occurring protein.

The polycation may be an animal protein, such as collagen and gelatin. In some embodiments, the polycation is not gelatin, and may not be an animal protein.

In some embodiments, the polycation is selected from a polysaccharide, a plant protein and a milk protein. In this way, the polycation may be vegetarian. In some embodiments, the polycation is not derived from porcine or fish sources.

In some embodiments, the polycation is selected from a polysaccharide and a plant protein. In these embodiments, the polycation is obtained from a non-animal source, and is thus vegan.

A plant protein is a protein obtained or obtainable from plant sources, or a derivative thereof. Examples of plant proteins include vegetable proteins. A plant protein may be a protein isolate obtainable from, or obtained from a plant, such as a vegetable. The protein may be isolated from one or more parts of a plant selected from fruit, root, rhizome, stem and leaf.

A milk protein is a protein derivable from milk or a derivative thereof. A milk protein may be a protein isolate obtainable from, or obtained from milk, such as animal milk, such as cow, sheep, or goat milk.

Preferably, the polycation is a polysaccharide. More preferably, the polycation is chitosan. The chitosan may be of animal or fungal origin. Preferably, the chitosan is of fungal origin.

The chitosan may have a molecular weight of 10 kDa or more, such as 50 kDa or more, such as 100 kDa or more. The chitosan may have a molecular weight that is no more than 500 kDa, such as no more than 250 kDa, such as no more than 200 kDa, such as no more than 150 kDa, such as no more than 125 kDa, such as no more than 100 kDa. Preferably, the chitosan is of low molecular weight, such as 150 kDa or less, such as around 100 kDa.

A polyanion is an anionic polymer. By “anionic polymer”, it is meant a polymer having a net negative charge. In the complex coacervate, the polyanion may be the counterion to the polycation.

The polyanion may be a synthetic polymer, or the polyanion may be a naturally occurring polymer. Examples of naturally occurring polymers as the polyanion include cellulose and its derivatives, starch and gums.

The polyanion may be selected from cellulose, starch, modified starch, agar, alginate, xanthan gum, plant gums, casein, zein, agarose, polyvinyl alcohol, isosorbide, derivatives thereof and salts thereof.

The polyanion may be a plant gum. Preferred examples include acacia gum, which may also be referred to as gum Arabic.

The polyanion may be provided as a salt, or may be provided as a free acid. When the polyanion is a salt, the counterion to the ionic form of the polyanion may be a metal ion or an organic ion. For example, the counter ion may be selected from sodium, potassium and calcium. Examples of the salt as the polyanion include sodium caseinate.

Derivatives of the polyanion include alkyl derivatives and substituted alkyl derivatives. In some embodiments, the derivative is an alkyl or substituted alkyl derivative of cellulose. In some embodiments, the cellulose derivatives are selected from methylcellulose, hydroxypropyl cellulose, and carboxymethyl cellulose. Of these, carboxymethyl cellulose may be preferred.

The polyanion may be selected from carboxymethylcellulose, starch, modified starch, agar, alginate, xanthan gum, plant gums, sodium caseinate, zein, agarose, polyvinyl alcohol, and isosorbide.

Preferably, the polyanion is selected from plant gums and carboxymethylcellulose. In some embodiments, the polyanion is a plant gum, and preferably is acacia gum. In some embodiments, the polyanion is carboxymethylcellulose.

In some embodiments, the polycation is chitosan and the polyanion is selected from acacia gum and carboxymethyl cellulose. In some embodiments, the polycation is chitosan and the polyanion is acacia gum. In other embodiments, the polycation is chitosan and the polyanion is carboxymethyl cellulose.

The polycation and the polyanion may electrically cancel each out. Alternatively, the polycation and the polyanion do not need to cancel each other out. In some embodiments, one or both of the polycation and the polyanion may comprise a counterion other than the other of the polycation or the polyanion.

The ratio of the polyanion to the polycation provided in step (i) is not particularly limited. The amount of the polyanion may be 1 part or more by weight relative to 1 part by weight of the polycation, such as 2 parts or more, such as 3 parts or more, such as 4 parts or more, such as 5 parts or more, such as 6 parts or more. The amount of the polyanion may be 20 parts or less by weight relative to 1 part by weight of the polycation, such as 15 parts or less, such as 10 parts or less, such as 9 parts or less, such as 8 parts or less, such as 7 parts or less. The amount of the polyanion may be in a range with lower and upper values given above, such as 1 to 20 parts by weight relative to 1 part by weight of the polycation.

The complex coacervate provided in step (i) may be in the form of droplets in the aqueous phase. These droplets may aggregate around the organic phase that is dispersed in step (ii), such as to encapsulate the organic phase during step (iii).

The complex coacervate in a microcapsule prepared according to the method of the present invention may be in the form of a coacervate shell, which may be provided at the interface between the organic phase and the aqueous phase.

The ratio of the polyanion to the polycation in the microcapsule shell is not particularly limited. The ratio of the polyanion to the polycation in the microcapsule shell may be substantially the same (and is generally expected to be the same) as the relative ratios of these components provided in step (i). For example, the ratio may be selected from the values and ranges given above.

### Polyisocyanate

The microcapsule of the present invention comprises a polymer obtained by interfacial polymerisation of a polyisocyanate. In some embodiments, the polymer is a polyurethane.

The polyisocyanate may be oil soluble. In these embodiments, the polyisocyanate may be provided as a solution in the organic phase.

The polyisocyanate may be a polyiso(thio)cyanate. That is, the polyisocyanate may be selected from a polyisocyanate and a polyisothiocyanate.

The polyisocyanate may comprise one or more aromatic groups such as phenyl, toluyl, xylyl and naphthyl.

The polyisocyanate may be an aromatic polyisocyanate. By “aromatic polyisocyanate”, it is meant a polyisocyanate having two or more isocyanate groups that are bonded directly to an aromatic moiety.

The polyisocyanate may be an aliphatic polyisocyanate. By “aliphatic polyisocyanate”, it is meant a polyisocyanate having two or more isocyanate groups that are bonded directly to an aliphatic moiety. The aliphatic moiety may be an alkyl group or an alkylene group, which may be optionally substituted. In some of these embodiments, the aliphatic polyisocyanate may further comprise an aromatic moiety. In other embodiments, the aliphatic polyisocyanate does not comprise an aromatic moiety.

Preferably, the polyisocyanate is an aliphatic polyisocyanate comprising an aromatic group.

Preferably, the polyisocyanate is selected from one or more of xylylene diisocyanate (XDI), hydrogenated xylylene diisocyanate (HXDI), toluene diisocyanate, hexamethylene diisocyanate (HDI), pentamethylene diisocyanate, methylene diphenyl diisocyanate, an adduct thereof and an oligomer thereof. One or more of the polyisocyanates above may be provided in the organic phase in step (ii).

An adduct of a polyisocyanate may be a trimethylol-propane adduct.

An oligomer of a polyisocyanate may include dimers, trimers, uretdionea, isocyanuratea, iminooxadiazinediones, allophanates and biurets.

In a preferred embodiment, the polyisocyanate is an adduct of xylylene diisocyanate (XDI), such as trimethylol-propane adduct of xylylene diisocyanate (XDI).

In the microcapsule shell, the polymer product of the interfacial polymerisation may be bonded to the coacervate. Without wishing to be bound be theory, it is thought that the coacervate participates in the polymerisation reaction with the polyisocyanate. The polymer may be present as a shell around the encapsulated organic phase, which may be present as an inner shell that is held within a coacervate outer shell. The polymer shell and the coacervate shell may be covalently bonded. The polymer and the coacervate may be interwoven to form a polymeric network. One or both of the coacervate and the polymer product in the microcapsule shell may be crosslinked. For example, two or more polymers selected independently from the polycation, the polyanion and the polymer product may be crosslinked by reaction with the crosslinking agent.

### Crosslinking Agent

In the methods of the present invention, a crosslinking agent is provided in step (iv).

A crosslinking agent is a compound able to crosslink a polymer, such as by forming covalent bonds to one or more polymer chains. The crosslinks may be intramolecular, or they may be intermolecular, and both may be present.

The crosslinking agent may be selected from one or more of glutaraldehyde, formaldehyde, acetaldehyde, glyoxal, epichlorohydrin, transglutaminase, tannic acid, sodium tripolyphosphate, boric acid, genipin, N-(3-dimethylaminopropyl)-N′-ethylcarbodiimide hydrochloride, bis(triethoxysilylpropyl) tetrasulfide, vinyltriethoxysilane, vinyltrimethoxysilane, dimethyl suberimidate, 1,2-cyclopentanedione and 1,3-cyclopentanedione.

The crosslinking agent may comprise one or more aldehyde groups. The crosslinking agent may be selected from glutaraldehyde, acetaldehyde, glyoxal and formaldehyde. These crosslinking agents have good crosslinking activity under acidic conditions, under which the coacervation can also proceed.

Preferably, the crosslinking agent is glutaraldehyde.

In some embodiments, the crosslinking agent is an enzyme, such as transglutaminase.

In some embodiments, the crosslinking agent is selected from glutaraldehyde, formaldehyde, acetaldehyde, glyoxal, epichlorohydrin and transglutaminase.

The steps (iii) and (iv) may be performed in order, so that the crosslinking agent is added to the dispersion of step (iii). In other embodiments, the steps (iii) and (iv) may be performed simultaneously.

The weight of the dispersion includes the aqueous phase, the organic phase, and the reagents that are dissolved or mixed in either phase during the preceding steps, such as the steps (i), (ii) and (iii).

The amount of crosslinking agent added during step (iv) influences the characteristics of the microcapsule. If the amount of the crosslinking agent is too low, the stability and/or the performance of the microcapsules may be affected. The microcapsules may not meet stability and performance requirements when the amount of the crosslinking agent used is too low, particularly when the encapsulated cargo comprises small volatile compounds. If the amount of the crosslinking agent used is too high, the microcapsule is not biodegradable. This is shown in the examples below, whereby microcapsules produced by crosslinking with no more than 0.03 wt. % of a crosslinking agent are shown to be inherently biodegradable according to the OECD 301F test. In contrast, microcapsules produced using a higher amount of a crosslinking agent is not inherently biodegradable by the same test.

Preferably, the amount of the crosslinking agent added in step (iv) provides a balance between the stability of the microcapsules and the biodegradability of the microcapsules. The preferred amounts of the crosslinking agent are set out below, with the most preferred amounts providing microcapsules with increased biodegradability whilst maintaining good stability and performance.

The amount of the crosslinking agent added may be up to 0.5% by weight relative to the dispersion, such as up to 0.4%, such as up to 0.3%, such as up to 0.2%, such as up to 0.1%, such as up to 0.09%, such as up to 0.08%, such as up to 0.07%, such as up to 0.06%, such as up to 0.05%, such as up to 0.04%, such as up to 0.03%, such as up 0.02%, such as up to 0.015%, such as up to 0.01%, such as up to 0.009%, such as up to 0.008%. Preferably, the amount of the crosslinking agent added is up to 0.03% by weight relative to the dispersion.

The amount of the crosslinking agent may be 0.0001% or more by weight of the dispersion, such as 0.001% or more, such as 0.002% or more, such as 0.003% or more, such as 0.004% or more, such as 0.005% or more, such as 0.006% or more, such as 0.007% or more, such as 0.008% or more, such as 0.009% or more, such as 0.01% or more.

In some embodiments, the amount of the crosslinking agent may be in a range with lower and upper values given above, such as 0.0001% to 0.5% by weight of the dispersion. Preferably, the amount of the crosslinking agent is 0.001% to 0.03% by weight of the dispersion, and more preferably 0.001% to 0.02%.

The amount of the crosslinking agent added may be 0.0001% or more by weight relative to the weight of water in the dispersion, such as 0.001% or more, such as 0.002% or more, such as 0.003% or more, such as 0.004% or more, such as 0.005% or more, such as 0.006% or more, such as 0.007% or more, such as 0.008% or more, such as 0.009% or more, such as 0.01% or more, such as 0.015% or more.

The amount of the crosslinking agent added may be up to 0.5% by weight relative to the weight of water in the dispersion, such as up to 0.4%, such as up to 0.3%, such as up to 0.2%, such as up to 0.1%, such as up to 0.09%, such as up to 0.08%, such as up to 0.07%, such as up to 0.06%, such as up to 0.05%, such as up to 0.04%, such as up to 0.03%, such as up 0.02%, such as up to 0.015%, such as up to 0.01%.

Preferably, the amount of the crosslinking agent is up to 0.03% by weight relative to the weight of water in the dispersion, and more preferably up to 0.02%.

In some embodiments, the amount of the crosslinking agent may be in a range with lower and upper values given above, such as 0.0001% to 0.5% by weight of relative to the weight of water in the dispersion, such as 0.001% to 0.03%.

The amount of the crosslinking agent added to the dispersion may be 0.5 parts by weight or more relative to 1,000 parts by weight of the complex coacervate provided in step (i), such as 1 part or more by weight, such as 2 parts or more, such as 3 parts or more, such as 4 parts or more. The amount of the crosslinking agent added to dispersion may be 100 parts or less by weight relative to 1,000 parts by weight of the complex coacervate provided in step (i), such as 50 parts or less, such as 25 parts or less, such as 20 parts or less, such as 15 parts or less, such as 10 parts or less, such as 9 parts or less, such as 8 parts or less, such as 7 parts or less, such as 6 parts or less, such as 5 parts or less, such as 4 parts or less, such as 3 parts or less. The amount of the crosslinking agent added to the dispersion may be in a range with lower and upper values given above, such as 0.5 to 100 parts by weight relative to 1,000 parts by weight of the complex coacervate provided in step (i).

In additional embodiments, the amount of the crosslinking agent added to the dispersion may be from 0.5 to 10 parts by weight relative to 1,000 parts by weight of the complex coacervate provided in step (i), such as from 0.5 to 8 parts, such as from 0.5 to 7 parts, such as from 0.5 to 6 parts, such as from 0.5 to 5 parts, such as from 0.5 to 4 parts, including 1 to 8 parts, such as 1 to 7 parts, such as 1 to 6 parts, such as 1 to 5 parts, such as 1 to 4 parts.

The amount of the crosslinking agent added to the dispersion may be 1 mole equivalent or more of the polycation in step (i), such as 5 mole equivalents or more, such as 10 mole equivalents or more, such as 15 mole equivalents or more, such as 20 mole equivalents or more, such as 25 mole equivalents or more, such as 30 mole equivalents or more. The amount of the crosslinking agent added to the dispersion may be up to 200 mole equivalents of the polycation in step (i), such as up to 150 mole equivalents, such as up to 100 mole equivalents, such as up to 75 mole equivalents, such as up to 50 mole equivalents, such as up to 40 mole equivalents, such as up to 30 mole equivalents, such as up to 25 mole equivalents. The amount of the crosslinking agent added to the dispersion may be in a range with lower and upper values given above, such as 1 to 200 mole equivalents of the polycation.

The amount of the crosslinking agent added to the dispersion may be 0.1 parts or more by weight relative to 100 parts by weight of the polyisocyanate added in step (ii), such as 0.5 parts or more, such as 1 part or more, such as 2 parts or more. The amount of the crosslinking agent added to the dispersion may be 20 parts or less relative to 100 parts by weight of the polyisocyanate added in step (ii), such as 10 parts or less, such as 5 parts or less, such as 4 parts or less, such as 3 parts or less, such as 2 parts or less. The amount of the crosslinking agent added to the dispersion may be in a range with lower and upper values given above, such as 0.1 to 20 parts by weight of the polyisocyanate added in step (ii).

The amount of the crosslinking agent added to the dispersion may be 0.001 mole equivalents or more of the polyisocyanate added in step (ii), such as 0.01 mole equivalents or more, such as 0.05 mole equivalents or more, such as 0.1 mole equivalents or more. The amount of the crosslinking agent added to the dispersion may be up to 2 mole equivalents of the polyisocyanate added in step (ii), such as up to 1 mole equivalents, such as up to 0.5 mole equivalent, such as up to 0.2 mole equivalents, such as up to 0.15 mole equivalents. The amount of the crosslinking agent added to the dispersion may be in a range with upper and lower values given above, such as 0.001 to 2 mole equivalents of the polyisocyanate added in step (ii).

### Organic Phase

The organic phase dispersed in the aqueous phase during step (ii) comprises the polyisocyanate.

The encapsulated organic phase in the microcapsule may be substantially depleted of polyisocyanate, such as after inducing polymerisation during step (iii), or after introducing the crosslinking agent during step (iv). The encapsulated organic phase may comprise a polyisocyanate, and this may be polyisocyanate that has not been consumed in the preparation of the microcapsule shell.

The organic phase may also comprise one or more of a fragrance, a cosmetic active, a malodour counteractive, an antimicrobial agent and an insecticide.

The organic phase preferably comprises a fragrance. A fragrance may comprise a fragrance compound or composition having suitable odorant properties for use as a perfume.

Fragrance compounds may include alcohols, aldehydes, esters, ketones, lactones, thiols, terpenes and essential oils.

In some embodiments the organic phase comprises an essential oil. Examples include oils that are obtained or obtainable from lemon, orange, peppermint, clove, cinnamon, cedar, eucalyptus, jasmine, patchouli, sandalwood, bergamot, grapefruit, lavender, tea tree, and cedar wood.

The fragrance that is encapsulated may be AR772948 (CPL Aromas) or AR775855 (CPL Aromas). The components of these fragrances are shown in Table 1.

TABLE 1
Fragrance compositions

| AR772948 | AR775855 |
|---|---|
| 5-Heptyloxolan-2-one | Undec-10-enal |
| 5-Pentyloxolan-2-one | 2-Methylundecanal |
| Prop-2-enyl 3-cyclohexylpropanoate | 5-Pentyloxolan-2-one |
| 1-(2,6,6-Trimethyl-2-cyclohexen-1-yl)hepta- | Allyl heptanoate |
| 1,6-dien-3-one | |
| 3a,4,5,6,7,7a-Hexahydro-1H-4,7- | Methyl 2-[(7-hydroxy-3,7- |
| methanoinden-1-yl acetate | dimethyloctylidene)amino]benzoate |
| 3a,4,5,6,7,7a-Hexahydro-4,7-methano-1H- | 1-(2,6,6-Trimethylcyclohex-3-en-1-yl)but-2- |
| inden-6-yl propionate | en-1-one |
| Allyl(cyclohexyloxy)acetate | Bis(2-ethylhexyl) adipate |
| Bis(2-ethylhexyl) adipate | 1-(5,5-Dimethylcyclohex-1-en-1-yl)pent-4- |
| | en-1-one |
| 1-(5,5-Dimethylcyclohex-1-en-1-yl)pent-4- | 1,3,3-Trimethyl-2-oxabicyclo[2.2.2]octane |
| en-1-one | |
| Ethyl-2-methylbutanoate | 1,7,7-Trimethylbicyclo[2.2.1]hept-2-yl |
| | acetate |
| 1,3,3-trimethyl-2-oxabicyclo[2.2.2]octane | Ethyl-2-methylpentanoate |
| Hex-3-en-1-yl acetate | 1-Phenylethyl acetate |
| 1,7,7-Trimethylbicyclo[2.2.1]hept-2-yl | 2,4-Dimethylcyclohex-3-ene-1- |
| acetate | carbaldehyde |
| Ethyl-2-methylpentanoate | |
| 1-(4-Methylphenyl)ethanone | |
| Methyl 2-aminobenzoate | |
| 1-(2,6,6-Trimethylcyclohex-1-en-1-yl)pent-1- | |
| en-3-one | |
| 2-Methoxynaphthalene | |
| 1-(2-Naphthyl)ethanone | |
| 2,4-Dimethylcyclohex-3-ene-1-carbaldehyde | |
| (E)-4-Methyldec-3-en-5-ol | |
| 2-tert-Butylcyclohexyl acetate | |
| 4-tert-Butylcyclohexyl acetate | |

The organic phase may comprise one or more materials selected from:
aldehydes, such as 2-methyldecanal, undecanal, dodecanal, 2-methylundecanal, and 2-benzylideneheptanal;
esters such as decyl acetate, nonyl acetate, prop-2-enyl 3-cyclohexylpropanoate, prop-2-enyl heptanoate, and 2,6-dimethylheptan-4-yl acetate;
ketones such as 1,6-dioxacycloheptadecan-7-one, 16-oxacyclohexadecan-1-one, 5-methyl-5-phenylhexan-3-one, 1-oxacycloheptadecan-2-one, and 4,7-dimethyloct-6-en-3-one;
alcohols such as1-(2-tert-butylcyclohexyl)oxybutan-2-ol, (1S,2R,5S,7R,8R)-2,6,6,8-tetramethyltricyclo[5.3.1.01,5]undecan-8-ol, 1-(2,2,6-trimethylcyclohexyl) hexan-3-ol, 4-tert-butylcyclohexan-1-ol, and 3,7,11-trimethyldodeca-1,6,10-trien-3-ol;
nitriles such as tetradecanenitrile, dodecanenitrile, 3-methyldodecanonitrile, (3Z)-trideca-3,12-dienenitrile, and tridec-2-enenitrile;
alkenes such as undeca-1,3,5-triene, 1-methyl-4-prop-1-en-2-ylcyclohexene, 2-butoxyethylbenzene, phenylmethoxymethylbenzene, and 3,7,7-trimethylbicyclo[4.1.0]hept-3-ene;
aryls such as 2-methoxyethylbenzene, 1-methyl-4-propan-2-ylbenzene, 2-propan-2-yloxyethylbenzene, 2′,2′,8′,8′-tetramethylspiro[1,3-dioxolane-2,10′-octahydro-1H-2,4a-methanonapthalene]; and
heteroaryls such as 4,5,6,7,8,9,10,11,12,13-decahydrocyclododeca[d][1,3]oxazole, and 4-[(3E)-4,8-dimethylnona-3,7-dienyl]pyridine.

The above materials, for example, have been found to be stable in the microcapsules of the present invention.

The organic phase may comprise one or more solvents selected from bis(2-ethylhexyl) adipate, diethyl benzene-1,2-dicarboxylate, propan-2-yl tetradecanoate, Helianthus annuus seed oil, Prunus amygdalus dulcis oil, Ricinus communis oil, caprylic acid triglyceride, capric acid triglyceride, octanoic acid, decanoic acid, mineral oil, shea butter and benzyl benzoate.

In additional embodiments, the organic phase may comprise one or more solvents selected from bis(2-ethylhexyl) adipate, diethyl benzene-1,2-dicarboxylate, propan-2-yl tetradecanoate, triethyl 2-hydroxypropane-1,2,3-tricarboxylate, Helianthus annuus seed oil, Prunus amygdalus dulcis oil, Ricinus communis oil, caprylic acid triglyceride, capric acid triglyceride, octanoic acid, decanoic acid, mineral oil, benzyl benzoate, and a butter or wax such as shea butter, bees wax and stearic acid.

The organic phase comprising the polyisocyanate may be prepared by admixing the components.

### Microcapsule

The microcapsule of the present invention comprises a microcapsule shell. The microcapsule shell defines an internal volume and encapsulates an organic phase. The microcapsule shell may form a barrier that limits or prevents the release of the components of the encapsulated organic phase, and in particular volatile components.

The microcapsule shell comprises a coacervate and a polymer, such as a polyurethane. For example, the polymer may be provided as a shell surrounding the organic phase, and may be provided as an inner shell. The coacervate may be provided as a shell at the boundary of the aqueous phase, and may be provided as an outer shell. The polymer and the coacervate may be interwoven in a polymeric network.

The microcapsule may be spherical in shape, or substantially spherical. The microcapsule may be non-spherical, such as ellipsoidal.

A microcapsule may be characterised by the thickness of the microcapsule shell.

A population of microcapsules may be defined by an average microcapsule shell thickness. By “average microcapsule shell thickness”, it is meant the mean value of the shell thickness of individual microcapsules in a collection of microcapsules. The population of microcapsules may be a typical amount that can be analysed together such as by microscopy, such as 100 microcapsules.

A microcapsule may be characterised by its largest dimension. By “largest dimension”, it is meant the maximal diameter in the largest cross-section of the microcapsule, such as the diameter of the microcapsule.

A population of microcapsules may be defined by an average largest dimension. By “average largest dimension”, it is meant the mean value of the largest dimension of individual microcapsules in a collection of microcapsules, where the largest dimension is as defined above, such as the diameter. The population of microcapsules may be a typical amount that can be analysed together such as by microscopy, such as 100 microcapsules.

The thickness and the largest dimension, such as diameter, of a microcapsule may be measured by optical microscopy. These values may be determined using a Microtec RM-1-MET microscope at 20× zoom, using the software Infinity Analyze.

In one embodiment, the organic phase to be encapsulated is dispersed in the aqueous phase with mixing. The speed of mixing is selected so as to provide microcapsules having a desired largest dimension and/or a desired microcapsule shell thickness. For example, a higher speed of mixing may be selected to obtain microcapsules having a smaller average dimension. The thickness of the microcapsule shells may also be affected by the speed of mixing, such as by affecting the shear of the system.

The microcapsule shell thickness may also be controlled by the concentration of the polyisocyanate and/or the coacervate that is provided in the dispersion.

The physical and chemical properties of the organic phase may also affect the size, thickness or morphology of the microcapsules, particularly when the organic phase comprises a fragrance. However, due to the complexity of fragrances, the effect of the fragrance may be difficult to predict.

The microcapsule may have a wall thickness that is 0.001 μm or more, such as 0.01 μm or more, such as 0.1 μm or more, such as 1 μm or more, such as 5 μm or more, such as 10 μm, such as 15 μm or more, such as 20 μm or more, such as 25 μm or more. The microcapsules may have a wall thickness that is 200 μm or less, such as 150 μm or less, such as 100 μm or less, such as 50 μm or less, such as 25 μm or less.

The microcapsule may have a wall thickness in a range with the lower and upper values selected from the above, such as 0.01 μm to 200 μm, such as 0.01 μm to 50 μm.

A population of microcapsules may have an average wall thickness as above. Preferably, a population of microcapsules have an average wall thickness of 1 μm to 20 μm, such as 1 μm to 10 μm, such as 5 μm to 10 μm.

The microcapsules may have a largest dimension that is 0.01 μm or more, such as 0.1 μm or more, such as 1 μm or more, such as 5 μm or more, such as 10 μm, such as 20 μm or more, such as 30 μm or more. The microcapsules may have a largest dimension that is 1,200 μm or less, such as 1,000 μm or less, such as 800 μm or less, such as 600 μm or less, such as 400 μm or less, such as 200 μm or less, such as 150 μm or less, such as 100 μm or less, such as 50 μm or less, such as 40 μm or less, such as 30 μm or less, such as 25 μm or less, such as 20 μm or less.

The microcapsules may have a largest dimension in a range with the lower and upper values selected from the above, such as 0.1 μm to 1,200 μm. Preferably, the microcapsule has a largest dimension of 0.1 μm to 200 μm, more preferably of 0.1 μm to 150 μm, most preferably of 1 to 120 μm.

A population of microcapsules may have an average largest dimension as above. Preferably, a population of microcapsules have an average largest dimension of 0.1 μm to 100 μm, such as 1 μm to 50 μm.

The microcapsule of the present invention may be biodegradable.

The biodegradability of the microcapsule may be defined according to the OECD test guidelines. For example, the microcapsule may pass one or more of the OECD TG 301 B, C, D, and F tests. The OECD TG 301 tests are methods designed to screen ready biodegradability in aqueous media. Preferably, respirometric test such as the OECD TG 301B and the OECD TG 301F tests can be used as these are compatible with testing insoluble materials. OECD TG 301B measures the CO₂ production (Theoretical CO₂ production, ThCO₂) of an inoculum that is introduced to the test substance, whilst OECD TG 301F measures the oxygen uptake (Theoretical Oxygen Demand, ThOD) of a microbial population during biodegradation of the test substance. Using the OECD TG 301F test for example, samples are classified as readily biodegradable when 60% ThOD is achieved in a 10-day window. Samples achieving 60% ThOD between 10 days and 28 days can be classified as inherently biodegradable. The biodegradability test may be performed on a sample of around 2,000 mg/L of a microcapsule slurry.

Preferably, the microcapsule passes the OECD TG 301B test or the OECD TG 301F test and is classified as inherently biodegradable or as readily biodegradable by one or more of these tests.

In some embodiments, the microcapsule is classified as inherently biodegradable by the OECD TG 301F and/or the OECD TG 301B test, such as the OECD TG 301F test.

In some embodiments, the microcapsule is classified as readily biodegradable by the OECD TG 301F test and/or the OECD TG 301B test, such as the OECD TG 301F test.

The biodegradability of the microcapsules may be defined according to the ISO standards, and the microcapsules may meet the criteria of ISO 17556, ISO 14851, ISO 14852, ISO 19679 and ISO 22404.

### Composition of Microcapsules

The microcapsule of the present invention may be provided in a composition. The composition may be suitable for use as a consumer product.

The composition comprises a plurality of microcapsules according to the present invention.

In some embodiments, the microcapsules may be suspended in the composition.

The composition may be an aqueous composition. For example, the microcapsule may be provided as a suspension in an aqueous phase.

The composition may be provided as part of a consumer product. A consumer product may be selected from a personal care product, a fabric care product, a home care product, a fragrance product and a textile product.

Examples of a personal care product include an antiperspirant cream, a antiperspirant spray, a deodorant, a roll on, a deodorant stick, a liquid shampoo, a liquid shower gel, a liquid hair conditioner, a solid shampoo, a solid shower gel, a solid hair conditioner, a body wash, a bath fragrance, a bath bomb, a bath salt, a soap, a soap bar, a hand wash, a hand sanitiser, and hand gel, a body cream, a hand moisturiser, a face moisturiser, a hair gel, a hair wax, a hair mousse, a hair dye, a hair bleach, a shaving cream, a shaving paste, a shaving powder, a face mask, a depilatory cream, and a fake tan.

Examples of a fabric care product include a scent booster, a liquid laundry detergent, a liquid fabric conditioner, a detergent powder, a fabric conditioner powder, a laundry detergent sheet, a fabric detergent sheet, a fabric refresher, a laundry prewash, a laundry aid, an ironing water, and a leather care product.

Examples of a home care product include a furniture polish, a floor cleaner, a window cleaner, a surface cleaner, an all-purpose cleaner, a carpet shampoo, a bleach, a toilet cleaner, a toilet block, a urinal cake, a de-scaler, a disinfectant, a washing up liquid, a solid washing up soap, a dishwasher tablet, a dishwasher powder, and a dishwasher sheet.

Examples of a fragrance product include a diffuser, a candle, an air refresher, a reed diffuser, a incense stick, a car wash, a fine fragrance product, an Eau De Toilette, an Eau de Parfum, a cologne, a solid perfume, a perfume gel, and a scratch and sniff card.

Examples of a textile product include a padded textile, and a clothing item.

Preferably, the consumer product is selected from a liquid laundry detergent, a liquid fabric conditioner, a detergent powder, a fabric conditioner powder, a laundry detergent sheet, a fabric detergent sheet, a fabric refresher, a laundry prewash, and a laundry aid.

The composition may further comprise one or more of surfactants, preservatives, pH stabilisers, thickening agents, opacifiers, sequestrants, propylene glycol and salts. The base formation may further comprise a solvent, preferably water.

Examples of surfactants include quat surfactants, anionic surfactants and non-ionic surfactants.

The composition may be a fabric care composition comprising a microcapsule according to the present invention and a base formulation. The fabric care composition may be selected from a liquid fabric conditioner and a liquid laundry detergent. In these embodiments, the encapsulated organic phase may comprise a fragrance.

### Method

The invention provides a method for producing a microcapsule, comprising the steps of:
(i) providing a complex coacervate of a polycation with a polyanion in an aqueous phase;
(ii) dispersing an organic phase comprising a polyisocyanate in the aqueous phase;
(iii) inducing encapsulation of the organic phase by the complex coacervate, together with polymerising the polyisocyanate at the interface of the organic phase; and
(iv) adding a crosslinking agent to the dispersion of step (iii) in an amount of up to 0.03% by weight of the dispersion and permitting the crosslinking agent to form crosslinks.

The invention also provides a method for producing a microcapsule, comprising the steps of:
(i) providing a complex coacervate of a polycation with a polyanion in an aqueous phase;
(ii) dispersing an organic phase comprising a polyisocyanate in the aqueous phase;
(iii) inducing encapsulation of the organic phase by the complex coacervate and polymerising the polyisocyanate at the interface of the organic phase; and
(iv) adding a crosslinking agent to the dispersion of step (iii) in an amount of up to 10 parts by weight relative to 1,000 parts by weight of the complex coacervate provided in step (i), and permitting the crosslinking agent to form crosslinks.

The steps (i) to (iv) may be performed in order.

One or more of the steps (i) to (iv) may be performed simultaneously. For example, the steps (i) and (ii) may be performed simultaneously, or the steps (iii) and (iv) may be performed simultaneously.

### Step (i)

In step (i), a mixture is provided comprising a complex coacervate of a polycation and a polyanion in an aqueous phase. The polycation may be selected from a polysaccharide, a plant protein and a milk protein. The polyanion may be selected from one or more of cellulose, starch, modified starch, agar, alginate, xanthan gum, plant gum, casein, zein, agarose, polyvinyl alcohol, isosorbide, derivatives thereof and salts thereof.

The mixture of step (i) may be obtainable by mixing the polycation and the polyanion in an aqueous phase to induce complex coacervation.

The aqueous phase may be water, such as deionised water.

The mixture may be obtained as part of step (i), such as by mixing the polycation and the polyanion, simultaneously or sequentially in an aqueous phase to induce complex coacervation. The mixture may be provided pre-formed.

In some embodiment, the step (i) comprises mixing the polycation and the polyanion in an aqueous phase. The polycation and the polyanion may be added simultaneously, sequentially or separately to the aqueous phase.

In step (i), the complex coacervate of the polycation with the polyanion may be partially formed. By “partially formed” it is meant an aqueous mixture in which some degree of liquid-liquid phase separation has occurred. For example, droplets of the complex coacervate may start to form in step (i) and separate from the aqueous phase. The droplets may be spherical, or the droplets may be non-spherical, such as ellipsoidal. Coacervation may continue to occur during one or more of the subsequent steps, such as steps (ii), (iii) and (iv). It is believed that the droplets of the coacervate that start to develop during step (i) then aggregate together to encapsulate the organic phase in step (iii), such as by forming a coacervate shell. Aggregation may continue in the step (iv).

Complex coacervation may be induced by adjusting the temperature of a mixture comprising the polycation and the polyanion, and/or by adjusting the pH of the mixture. The pH may be measured by a pH probe.

By “adjusting the pH”, it is meant adding a reagent to the mixture to increase or decrease the pH. In some embodiments, an acid is added to decrease the pH of the mixture. In some embodiments, a base is added to increase the pH of the mixture. Suitable acids include hydrochloric acid, acetic acid, citric acid, lactic acid and sulfuric acid. Suitable bases include triethylamine, potassium hydroxide and sodium hydroxide. The acid or bases may be added to the mixture as an aqueous solution.

In some embodiments, where the polycation is chitosan and the polyanion is acacia gum, complex coacervation may be induced in step (i) by adjusting the pH of the mixture to 1.5 or more, such as 1.7 or more, such as 1.9 or more. Complex coacervation may be induced by adjusting the pH to 2.5 or less, such as 2.1 or less, such as 1.9 or less. Complex coacervation may be induced by adjusting the pH within a range with lower and upper values selected from the above, such as pH 1.5 to 2.5. Preferably, complex coacervation is induced by adjusting the pH of the mixture to 1.9.

Providing a mixture of a complex coacervate in an aqueous phase during step (i) at a pH that is near, but not at the pH for inducing encapsulation of the organic phase in step (iii) facilitates aggregate of the coacervate more efficiently around the organic phase in step (iii). These pH conditions also encourage the polyisocyanate to react with the coacervate during polymerisation, and not just with the polyanion or water.

The coacervate may be provided as droplets or other morphologies in the aqueous phase, which may then aggregate around the organic phase in step (iii).

In some embodiments where the polycation is chitosan and the polyanion is carboxylmethyl cellulose, complex coacervation may be induced in step (i) by adjusting the pH to 2.0 or more, such as 2.5 or more, such as 3.0 or more, such as 3.5 or more. Complex coacervation may be induced by adjusting the pH to 5.0 or less, such as 4.5 or less, such as 4.0 or less, such as 3.5 or less, such as 3.0 or less. Complex coacervation may be induced by adjusting the pH within a range with lower and upper values selected from the above.

In some embodiments, the formation of the complex coacervate is induced in step (i) at a pH that is the same as that in step (iii). In other embodiments, formation of the complex coacervate in step (i) is induced at a lower pH than the pH used in step (iii), such 0.1 pH units lower, such as 0.2 pH lower, such as 0.5 pH lower, such as 1 pH lower, such as 1.5 pH units lower.

By “adjusting the temperature”, it is meant heating or cooling the mixture.

Complex coacervation may be induced at a temperature that is 10° C. or more, such as 15° C. such as 20° C. or more.

Complex coacervation may be induced at a temperature that is 95° C. or less, such as 90° C. or less, such as 80° C., such as 70° C., such as 60° C. or less, such as 50° C. or less, such as 40° C. or less, such as 30° C. or less. In these embodiments, the temperature of the reaction mixture may be adjusted by external heating.

Complex coacervation may be induced at a temperature in the range with the lower and upper temperatures selected from the values given above, such as 10° C. to 95° C., such as 15° C. to 30° C. Complex coacervation may be carried out at ambient temperature.

During complex coacervation, the mixture may be stirred, such as at a stirring speed of 200 RPM or more, such as 300 RPM or more, such as 400 RPM or more. The speed of stirring may be 1,000 RPM or less, such as 900 RPM or less, such as 800 RPM or less, such as 700 RPM or less. The speed of mixing may be in a range with upper and lower values given above, such as 200 RPM to 1,000 RPM, such as 400 RPM to 700 RPM.

For each of the steps (i) to (iv), stirring may be by an overhead stirrer, such as with a high-speed dispersion blade. Other types of propellers blades can also be used, including a Rushton turbine and a 4-bladed propeller.

The stirring may be coupled with a homogeniser for emulsification. For example, the homogeniser may be at a speed of 5,000 RPM to 20,000 RPM, such as 10,000 RPM.

Stirring of the coacervate can be carried out in this way at a fastest speed without entailing air. The mixing may also be performed under inert atmosphere to exclude air, which may be preferred when the encapsulant is vulnerable to oxidation.

### Step (ii)

In step (ii) an organic phase comprising a polyisocyanate is dispersed in an aqueous phase.

In some embodiments, the organic phase is dispersed into the aqueous phase after introduction of the coacervate of step (i).

In some embodiments, step (ii) is performed before step (i). In these embodiments, the organic phase may be dispersed in an aqueous solution comprising one of the polycation or the polyanion, followed by addition of the other one of the polycation or the polyanion to the dispersion. In these embodiments, the coacervate is provided after step (ii).

In some embodiments, the organic phase is dispersed in the aqueous phase simultaneously with introduction of a complex coacervate.

Without wishing to be bound by theory, it is believed that due to the high reactivity of polyisocyanates at low and high pH due to their electrophilic nature, as soon as the organic phase is dispersed in step (ii), the polyisocyanate may start to react. The polymerisation reaction is then accelerated after adding the crosslinking agent in step (iv), particularly when step (iv) is performed at elevated temperatures, such as around 50° C.

In some embodiments, the organic phase is dispersed in the aqueous phase, for example to form droplets or other morphologies of the organic phase within the continuous aqueous phase. In these embodiments, the interfacial polymerisation and the encapsulation by the complex coacervate is around a droplet of the organic phase.

The method for dispersing the organic phase into the aqueous phase is not particularly limited. The organic phase may be added to the water and emulsified whilst stirring. The rate of stirring may be controlled to read a desired average droplet size. The rate of stirring may be 1,000 RPM or more, such as 1,500 RPM or more, such as 2,000 RPM or more, such as 2,500 RPM, such as 3,000 RPM or more. The rate of stirring may be at a speed of 4,000 RPM or less, such as 3,500 RPM or less, such as 3,000 RPM or less, such as 2,500 RPM or less, such as 2,000 RPM or less. The rate of stirring may be in a range with upper and lower values selected from the above. Where the dispersion in step (iii) or step (iv) is stirred, the rate of stirring may also be independently selected from the values and ranges above.

Preferably, the rate of stirring in step (ii) is from 1,500 RPM to 3,000 RPM, such as 2,000 RPM to 2,500 RPM.

The rate of stirring may be varied during step (ii). For example, the step (ii) may comprise two or more stages with mixing at different speeds.

For each of the steps (ii) to (iv), the stirring may be continuous with a previous step, or the stirring may be changed, stopped or restarted relative to the stirring in the previous step.

The organic phase comprises a polyisocyanate, as described above. The amount of the polyisocyanate in the organic phase is not particularly limited. The amount of the polyisocyanate may be 1 part or more by weight relative to 100 parts by weight of the coacervate, such as 5 parts or more, such as 10 parts or more, such as 15 parts or more. The amount of the polyisocyanate may be 50 parts or less by weight relative to 100 parts of the coacervate, such as 25 parts or less, such as 20 parts or less, such as 19 parts or less, such as 18 parts or less. The amount of the polyisocyanate may be in a range with lower and upper values given above, such as 1 to 50 parts by weight relative to 100 parts by weight of the coacervate.

The amount of the polyisocyanate may be 0.1% or more by weight of the organic phase, such as 0.5% or more, such as 1% or more, such as 2% or more, such as 3% or more. The amount of the polyisocyanate may be 10% or less by weight of the organic phase, such as 5% or less, such as 2% or less. Preferably, the amount of the polyisocyanate is 1% to 10% by weight of the organic phase, more preferably 1.5% to 5% by weight of the organic phase.

If the amount of the polyisocyanate added in step (ii) is too high, the physical properties of the microcapsule, and the biodegradability of the microcapsule, may be affected. For example, the microcapsule may be too hard to rupture during use. Adding too little of the polyisocyanate may lead to a chemically unstable microcapsule.

In some embodiments, the organic phase may comprise a fragrance. The amount of the fragrance is not particularly limited, and is selected based on the intended use and with the intended fragrance intensity in mind.

The amount of the fragrance may be 10 parts or more by weight relative to 1 part by weight of the polyisocyanate, such as 25 parts or more, such as 50 parts or more. The amount of the fragrance may be 200 parts or less by weight relative to 1 part by weight of the polyisocyanate, such as 100 parts or less, such as 75 parts or less, such as 50 parts or less. The amount of the fragrance may be in a range with lower and upper values given above, such as 10 to 200 parts by weight relative to 1 part by weight of the polyisocyanate.

The amount of the fragrance may be 10 parts or more by weight relative to 10 parts by weight of the coacervate in step (i), such as 25 parts or more, such as 50 parts or more, such as 75 parts or more. The amount of the fragrance may be 200 parts or less by weight relative to 10 parts by weight of the coacervate in step (i), such as 150 parts or less, such as 100 parts or less, such as 50 parts or less. The amount of the fragrance may be in a range with lower and upper values given above, such as 10 to 200 parts by weight relative to 10 parts by weight of the coacervate in step (i).

The amount of the fragrance may be 10% or more by weight of the dispersion, such as 15% or more. The amount of the fragrance may be 50% or less by weight of the dispersion, such as 40% or less, such as 30% or less, such as 20% or less. The amount of the fragrance may be in a range with lower and upper values given above, such as 10% to 50% by weight of the dispersion. Preferably, the amount of the fragrance is 15% to 20% by weight of the dispersion.

Microcapsules having a higher fragrance content are more efficient and require less storage volume. If the amount of the fragrance is too high, agglomeration of the microcapsules may occur. Without wishing to be bound by theory, it is believed that at higher fragrance concentration, the entropy of the system decreases and the microcapsules are likely to touch and agglomerate.

Step (iii)

In step (iii), encapsulating the organic phase by the complex coacervate is induced together with permitting polymerisation of the polyisocyanate at the interface of the organic phase.

The encapsulation may occur at the boundary of the organic phase, such as a boundary between the organic phase and the aqueous phase.

The encapsulation may be by aggregation of the complex coacervate provided in step (i), such as to form a coacervate shell around the organic phase. For example, US 2019/0240124 and WO 2021/018947 describe providing a suspension of complex coacervate nodules in an aqueous vehicle, which are then deposited at an interface of the aqueous vehicle adjacent to a hydrophobic internal face to form a coacervate.

In some embodiments, the interfacial polymerisation of the polyisocyanate may proceed under the same conditions as for inducing encapsulation of the organic phase by the complex coacervate. In these embodiments, polymerisation may occur simultaneously with the encapsulation by the complex coacervate.

In some embodiments, the encapsulation in step (iii) is induced by heating or cooling the dispersion. In other embodiments, the interfacial polymerisation and/or the encapsulation in step (iii) occurs at ambient temperature.

In some embodiments, step (iii) is performed at a temperature of 10° C. or more, such as 15° C. or more, such as 20° C. or more.

In some embodiments, step (iii) is performed at a temperature of 95° C. or less, such as 90° C. or less, such as 80° C., such as 70° C., such as 60° C. or less, such as 50° C. or less, such as 40° C. or less, such as 30° C. or less.

In some embodiments, step (iii) is performed at a temperature in the range with the lower and upper temperatures selected from the values given above, such as 10° C. to 95° C., such as 15° C. to 30° C. Step (iii) may be performed at ambient temperature.

When the polycation is chitosan, and the second polyanion is a plant gum, such as acacia gum, heating of the dispersion may not be required and preferably step (iii) is performed at ambient temperature.

In some embodiments, the encapsulation in step (iii) is induced by adjusting the pH of the dispersion. The pH may be adjusted by adding a reagent to increase or to decrease the pH.

In step (iii), the pH of the dispersion may be 2.5 or more, such as 2.7 or more, such as 2.9 or more. In step (iii), the pH of the mixture may be 3.5 or less, such as 3.1 or less, such as 2.9 or less.

In step (iii), the pH may be in a range with the lower and upper values selected from above, such as 2.5 to 3.5.

When the polycation is chitosan, and the second polyanion is a plant gum, such as acacia gum, preferably in step (iii) the pH of the dispersion is adjusted to around 2.95. In these embodiments, the dispersion in step (iii) may be at ambient temperature.

In some embodiments, the pH of the dispersion is increased in step (iii), such as by addition of a base.

In step (iii) the dispersion may be stirred. The rate of stirring may be selected from the values or ranges given above in step (ii). In some embodiments, the rate of stirring during step (iii) may be lower than the rate of stirring in step (ii). In some embodiments, the rate of stirring during step (iii) is continuous from that in step (ii).

### Step (iv)

In step (iv) a crosslinking agent is added to the mixture in an amount of up to 0.03% by weight, relative to the total weight of the dispersion in step (iii), and the crosslinking agent is permitted to form crosslinks.

In alternative embodiments in step (iv) a crosslinking agent is added to the mixture in an amount of up to 10 parts by weight relative to 1,000 parts by weight of the complex coacervate provided in step (i), and the crosslinking agent is permitted to form crosslinks

The crosslinking agent may react with one or more of the components of the coacervate and the polymer to form crosslinks. This helps to strengthen the microcapsule shell.

In some embodiments, the step (iv) occurs after the step (iii). In these embodiments, the encapsulation of the organic phase, and/or polymerisation may continue to occur during step (iv) in the presence of the crosslinking agent.

In some embodiments, the step (iv) occurs simultaneously with the step (iii). In these embodiments, encapsulation of the organic phase, and/or polymerisation may occur at the same time as the crosslinking reaction.

The dispersion may be diluted between steps (iii) and (iv), such as by adding water before the crosslinking agent is added. This helps to reduce agglomeration of the microcapsules during the crosslinking process.

The crosslinks formed during step (iv) may comprise covalent interactions between the crosslinking agent and one or more species in the dispersion, such as one or more polymers selected from the polycation, the polyanion and the polymerisation product of the polyisocyanate.

The temperature when the crosslinking agent is added to the dispersion may be below 30° C., such as between 0° C. and 30° C., such as between 15° C. and 30° C. The dispersion of step (iv) may be externally controlled at these temperatures. For example, the dispersion may be cooled or heated after a preceding step of the method to the above temperatures. Preferably, the crosslinking agent is added in step (iv) at ambient temperature.

The dispersion in step (iv) may be heated or cooled following addition of the crosslinking agent to the dispersion. Preferably, the dispersion is heated following addition of the crosslinking agent.

Step (iv) comprises a stage of permitting the crosslinking agent to react with one or more species in the dispersion. The stage of permitting the crosslinking agent to form crosslinks may be performed at a temperature that is 10° C. or more, such as 15° C. or more, such as 20° C. or more, such as 25° C. or more, such as 30° C. or more, such as 35° C. or more, such as 40° C. or more, such as 50° C. or more. The stage of permitting the crosslinking agent to form crosslinks may be performed at a temperature that is 90° C. or less, such as 80° C. or less, such as 70° C. or less, such as 60° C. or less, such as 50° C. or less. The temperature during the stage of permitting the crosslinking agent to form crosslinks may be performed may be in a range with lower and upper values given above, such as 10° C. to 90° C.

Preferably, the step (iv) comprises a stage of permitting the crosslinking agent to form crosslinks at a temperature of 30° C. or more, such as 40° C. or more, such as 50° C. or more. More preferably, the step (iv) comprises a stage of permitting the crosslinking agent to form crosslinks at a temperature of 30° C. to 60° C., and most preferably at a temperature of 30° C. to 50° C.

Step (iv) may comprise one or more stages of permitting the crosslinking agent to form crosslinks that are performed at different temperatures. For example, step (iv) may comprise two or more stages that are performed at different temperatures, such as three or more stages, following the addition of the crosslinking agent. The temperature that each stage is performed at may be selected independently from the values and ranges above.

Preferably, the dispersion in step (iv) is left at ambient temperature after adding the crosslinking agent, followed by a stage of heating the dispersion. In this way, changes in the pH of the dispersion during heating are reduced and the conditions are more favourable for encapsulation to occur.

Preferably, step (iv) comprises a stage of permitting the crosslinking agent to form crosslinks at ambient temperature, a stage performed at 40° C. or more, such as 40° C., and a stage performed at 50° C. or more, such as 50° C. In some embodiments, the above stages may be performed in order.

Without wishing to be bound by theory, a reaction temperature at ambient temperature reduces rapid changes in the pH of the reaction. A reaction temperature of about 40° C. facilitates crosslinking between the crosslinking agent and the coacervate. A reaction temperature of about 50° C. facilitates polymerisation of the polyisocyanate, which may occur in the stages (ii) and (iii) and is accelerated in stage (iv).

The reaction time during step (iv) may be 30 minutes or more, such as 1 hour or more. The reaction time during step (iv) may be 10 hours or less, such as 6 hours or less, such as 5 hours or less, such as 4 hours or less, such as 3 hours or less.

The reaction time during step (iv) may be a range selected from the lower and upper values given above, such as 30 minutes to 10 hours.

In some embodiments where the step (iv) comprises two or more stages performed at different temperatures, the reaction time at each temperature may independently be 5 minutes or more, such as 15 minutes or more, such as 30 minutes or more, such as 1 hour or more, such as 2 hours or more, such as 3 hours or more. The reaction time at each temperature may independently be 5 hours or less, such as 3 hours or less, such as 2 hours or less, such as 1.5 hour or less, such as 1 hour or less. The reaction time at each temperature may be a range selected from the values given, such as 5 minutes to 5 hours.

The crosslinking reaction in step (iv) may be deemed complete when the crosslinking agent and/or the polyisocyanate is substantially depleted from solution. This may be followed by FTIR, for example.

In some embodiments, the step (iv) may comprise stirring of the dispersion. The rate of stirring may be selected from the values or ranges given above in step (ii). Preferably, the rate of stirring in step (iv) is lower than that in step (ii). The rate of stirring may be substantially unchanged throughout step (iv), or the rate of stirring may be varied during step (iv). Preferably, the rate of stirring in step (iv) is around 2,000 RPM.

### Additional Composition for Preparing a Microcapsule

Additionally, or alternatively, the invention also provides a composition for preparing a microcapsule. The composition comprises:
an aqueous phase comprising a polycation and a polyanion;
an organic phase comprising a polyisocyanate; and
a crosslinking agent,
   wherein the crosslinking agent is present at an amount of up to 0.03% by weight of composition.

The amount of crosslinking agent may in a fixed amount relative to the total (i.e. combined) weight of the polycation and polyanion. Therefore, the invention also provides a composition for preparing a microcapsule, the composition comprising:
an aqueous phase comprising a polycation and a polyanion;
an organic phase comprising a polyisocyanate; and
a crosslinking agent,
   wherein the crosslinking agent is present at an amount of up to 10 parts by weight relative to 1,000 parts by total weight of the polycation and the polyanion.

The polycation and the polyanion in the aqueous phase is for forming a complex coacervate, as described herein. The aqueous phase may thus comprise a complex coacervate of the polycation and the polyanion. For example, about 10% or more of the polycation and/or polyanion may be in a coacervate, such as 20% or more, such as 50% or more, such as 70% or more, such as 80% or more, such as 90% or more, such as 95% or more.

The organic phase may comprise droplets, or another morphology, which may be dispersed in the aqueous phase. The polycation and the polyanion may be at least partially in a coacervate. The coacervate may be in the form of droplets or another morphology, which may be dispersed in the aqueous phase or may aggregate to form a shell encapsulating a droplet of the organic phase, such as to form an outer shell. The polyisocyanate may be at least partially polymerised to form a polymer, which may form a shell at the boundary of the aqueous phase, such as an inner shell. The polymer and the coacervate may be interwoven in a polymeric network.

Preferred features of the components in the composition are as described herein including each of the coacervate, polycation, polyanion, polyisocyanate, crosslinking agent, aqueous phase and organic phase. For example, the amount of crosslinking agent may be as described and expressed by weight or by mole equivalents relative to:
the composition, where the composition may correspond to the dispersion in the methods described herein;
the weight of water in the composition, where the composition may correspond to the dispersion in the methods described herein;
the weight of a complex coacervate, where the weight of the complex coacervate corresponds to the total weight of polycation and the polyanion in the composition;
the polycation; and/or
the polyisocyanate.

Preferably, the amount of the crosslinking agent is 0.5 parts by weight or more relative to 1,000 parts by total weight of the polycation and polyanion, such as 1 part or more by weight, such as 2 parts or more, such as 3 parts or more, such as 4 parts or more. The amount of the crosslinking agent may be 100 parts or less by weight relative to 1,000 parts by total weight of the polycation and polyanion, such as 50 parts or less, such as 25 parts or less, such as 20 parts or less, such as 15 parts or less, such as 10 parts or less, such as 9 parts or less, such as 8 parts or less, such as 7 parts or less, such as 6 parts or less, such as 5 parts or less, such as 4 parts or less, such as 3 parts or less. The amount of the crosslinking agent may be in a range with lower and upper values given above, such as 0.5 to 100 parts by weight relative to 1,000 parts by total weight of the polycation and polyanion, including 0.1 to 10 parts by weight.

More preferably, the crosslinking agent is in an amount of up to 5 parts by weight relative to 1,000 parts by weight of the polycation and the polyanion, such as up to 4 parts by weight relative to 1,000 parts by total weight of the polycation and the polyanion, such as up to 3 parts by weight.

In some embodiments, the amount of the crosslinking agent added to the dispersion is from 0.5 to 10 parts by weight relative to 1,000 parts by weight of the complex coacervate provided in step (i), such as from 0.5 to 8 parts, such as from 0.5 to 7 parts, such as from 0.5 to 6 parts, such as from 0.5 to 5 parts, such as from 0.5 to 4 parts, including 1 to 8 parts, such as 1 to 7 parts, such as 1 to 6 parts, such as 1 to 5 parts, such as 1 to 4 parts.

### Use of Microcapsules

The microcapsules of the present invention may be used to encapsulate a cargo.

In one embodiment, the cargo is selected from one or more of a fragrance, a cosmetic active, a malodour counteractive, an antimicrobial agent, and an insecticide.

Other examples of the cargo include a bactericide, a fungicide, a pharmaceutical agent, and a vitamin.

The release of the encapsulated cargo in use requires disruption of the microcapsule shell.

The release of the encapsulated cargo may be achieved by rubbing the microcapsule against a substrate, such as a fabric, skin or an appliance such as an iron or a washing machine.

### OTHER EMBODIMENTS

Each and every compatible combination of the embodiments described above is explicitly disclosed herein, as if each and every combination was individually and explicitly recited. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

### Results and Discussion

### Microcapsules

A general process 1 for producing microcapsules is as follows:
1. Polycation, such as chitosan, was dispersed in 300 ml water.
2. Polyanion, such as acacia gum, was dissolved in the above solution.
3. The pH was lowered to 1.9 using acid, such as 20% aq. HCl.
a. Polycation dissolves and the solution goes clear.
4. pH increased to 2.6 dropwise (1 per 10 seconds) using base, such as 5% aq. TEA.
a. Solution goes cloudy and coacervate starts to form.
5. In a separate beaker, polyisocyanate was added to fragrance.
6. The fragrance mixture was emulsified into the coacervate at 2,000 RPM.
7. Speed was lowered and pH was increased to 2.95 using base, such as 5% aq. TEA dropwise.
a. A coacervate shell should have formed around the fragrance.
8. 200 mL of additional water is added.
9. 50% aq. crosslinking agent, such as glutaraldehyde was added.
10. Left at RT for 1 hour.
11. Heated to 40° C. and left for 1.5 hour to accelerate/improve crosslinking.
12. Heated to 50° C. and left for 1 hour to induce/improve polymerisation.

A general process 2 for producing microcapsules is as follows:
1. Polycation, such as chitosan, was dispersed in water such as described in Table 2.
2. Polyanion, such as acacia gum, was dissolved in the above solution.
3. The pH was lowered to 1.9 using acid, such as 20% aq. HCl.
4. Polycation dissolves and the solution goes clear.
5. pH increased to 2.6 dropwise (1 per 10 seconds) using base, such as 5% aq. TEA.
6. Solution goes cloudy and coacervate starts to form.
7. In a separate beaker, polyisocyanate was added to fragrance.
8. The fragrance mixture was emulsified into the coacervate at 2,500 RPM.
9. Speed was lowered and pH was increased to 2.95 using base, such as 5% aq. TEA dropwise.
10. A coacervate shell should have formed around the fragrance.
11. 5% aq. crosslinking agent, such as glutaraldehyde was added.
12. Left at RT for 1 hour.
13. Heated to 40° C. and left for 3 hours to accelerate/improve crosslinking.
14. Heated to 50° C. and left for 1 hour to induce/improve polymerisation.

In both general processes, an overhead stirrer (Caframo BDC6015-220) with a high-speed dispersion blade was used for mixing the dispersions.

Samples 1 to 7 and additional Sample 10 were prepared according to the general process 1 or 2 above. The compositions are detailed in Table 2. 20% HCl, 5% TEA were provided as aqueous solutions, and polyisocyanate was provided in 75% ethyl acetate. The values correspond to the weight used of each solution. Glutaraldehyde was provided as either 5% or 50% aqueous solutions, and the values correspond to the total amount of glutaraldehyde added.

TABLE 2
Summary of microcapsule compositions

| | | Chitosan | | | | | | | | | | Polyisocyanate⁵ | | | | | |
|---|---|---|---|---|---|---|---|---|---|---|---|---|---|---|---|---|---|
| | | Kitozyme | Chibio | | Acacia | | 20% | | 5% | | | Takenate | Takenate | | | | |
| Sample | Process | CsH¹ | Mushroom² | | Gum³ | | HCl | | TEA | | Fragrance⁴ | D-110N | D-120N | Glutaraldehyde | | Water | |
| 1 | 1 | 3.6 g | — | | 24 | g | 6 | g | 31 | g | 112 g | 5.76 g | — | 1.5 | g | 500 | g |
| 2 | 1 | — | 3.6 | g | 24 | g | 6 | g | 31 | g | 112 g | 5.76 g | — | 1.5 | g | 500 | g |
| 3 | 1 | 3.6 g | — | | 24 | g | 6 | g | 31 | g | 112 g | — | 6.0 g | 1.5 | g | 500 | g |
| 4 | 1 | 3.6 g | — | | 24 | g | 6 | g | 31 | g | 112 g | 2.88 g | — | 1.5 | g | 500 | g |
| 5 | 1 | 3.6 g | — | | 24 | g | 6 | g | 31 | g | 112 g | 5.76 g | — | 0.075 | g | 500 | g |
| 6 | 1 | 1.5 g | — | | 10 | g | 2.8 | g | 19.25 | g | 112 g | 2.88 g | — | 0.05 | g | 500 | g |
| 7 | 2 | 1.5 g | — | | 10 | g | 2.8 | g | 19.25 | g | 112 g | 2.88 g | — | 0.05 | g | 500 | g |
| 10 | 2 | — | 1.63 | g | 10.82 | g | 4.4 | g | 31 | g | 180 g | 9.60 g | — | 0.05 | g | 362.5 | g |

¹KIOSMETINE-CSH, sourced from KitoZyme is derived from Agaricus bisporus. Degree of acetylation: 0-30% (mol %), viscosity: 12-25 cP
²Sourced from Qingdao Chibio Biotech Co., Ltd, derived from oyster mushrooms. Degree of acetylation: <2%, viscosity: 20-100 cP
³Type 8074 RD, sourced from Norevo GmbH
⁴AR772948, sourced from CPL Aromas
⁵75% solution in ethyl acetate, sourced from Mitsui Chemicals

Samples 1 to 4 are comparative examples using a higher concentration of glutaraldehyde.

Sample 2 is a comparative example using a different type of chitosan (from Chibio Mushroom) than for the other examples.

Sample 3 is a comparative example made using a more aliphatic polyisocyanate, the Takenate D-120N. Takenate D-120N is used in the same molar quantities as the D-110N. By using a more aliphatic isocyanate, it is expected for the capsules to be more biodegradable but less chemically stable.

Sample 4 is a comparative example using less polyisocyanate. Although, it is expected that the urethane moiety is biodegradable, decreasing the amount of polyisocyanate was expected to reduce the amount of crosslinking present in the interfacial polymerisation part of the shell.

Samples 5 to 7 and additional Sample 10 are embodiments of the invention. Samples 5 and 6 were prepared according to the general process 1. Sample 7 and 10 were prepared according to the general process 2.

An alternative general process 3 for producing microcapsules is as follows:
1. Polycation, such as chitosan, was dispersed in 500 ml water
2. Polyanion, such as D-isosorbide, was dissolved in the above solution.
3. The pH was lowered to 1.9 using acid, such as 20% aq. HCl.
4. Polycation dissolves and the solution goes clear.
5. pH increased to 5.3 dropwise (1 per 10 seconds) using base, such as 5% aq. TEA.
6. Solution goes cloudy and coacervate starts to form.
7. In a separate beaker, polyisocyanate was added to fragrance.
8. The fragrance mixture was emulsified into the coacervate at 2,500 RPM.
9. Speed was lowered and pH was increased to 7 using base, such as 5% aq. TEA dropwise.
10. A coacervate shell should have formed around the fragrance.
11. 5% aq. crosslinking agent, such as glutaraldehyde was added.
12. Left at RT for 1 hour.
13. Heated to 40° C. and left for 3 hours to accelerate/improve crosslinking.
14. Heated to 50° C. and left for 1 hour to induce/improve polymerisation.

Sample D was prepared according to general process 3.

### Microcapsule Analysis

Microcapsule were analysed using a Microtec RM-1-MET microscope at 20× zoom, using the Infinity Analyze software to determine the diameter and the shell thickness of microcapsules.

The microcapsules produced by either process 1 or process 2 have an average size of around 40 μm (range of 1 μm to 120 μm) and average thickness of 7 μm (minimum of 1 μm to 22 μm). However, the limit of detection was 1 μm and the wall thickness could be as low as 0.01 μm for smaller microcapsules.

### Comparative Microcapsules

Comparative Sample 8 prepared according to Example 2 of WO 2016/185171 were sourced from Lambson. The microcapsules of this comparative sample comprise chitosan (biomass derived N-acetylglucosamine/glucosamine copolymer having a molecular weight of 10 to 20 kDa) and acacia gum. The microcapsules do not comprise polyisocyanate. The microcapsules were crosslinked using around 0.21% by weight of glutaraldehyde with overnight stirring. The fragrance encapsulated was AR772948 (CPL Aromas).

The microcapsules of Comparative Sample 8 had a particle size range of 11 μm to 56 μm, with an average of 32 μm.

Comparative Sample 9 was prepared to the following method:
1. 5.09 g acacia gum (Type 8074 RD, Norevo GmbH) and 10.61 g fish Gelatine (240 bloom, Weishardt International, GeLiMa, a.s.) was dissolved in 508.4 g deionised water at 40° C. Mixing was done at 500 RPM using an overhead stirrer (Caframo).
2. The pH was decreased from 5.49 to 4.42 using 0.43 g 20% aq. HCl.
3. pH adjusted to 4.51 using 0.04 g 20% aq. NaOH.
4. The reaction was cooled to 33° C. to induce coacervation.
5. 0.13 g octanol was added to remove any induced air.
6. 6.19 g Takenate D-110N isocyanate was mixed into 70.53 g fragrance (AR772948, CPL Aromas).
7. The fragrance mixture was added to the water phase and emulsified at 2,000 RPM for 4 minutes.
8. Mixing was lowered to 700 RPM.
9. The reaction was cooled at approximately 0.5° C./minute to 14° C.
10. 0.44 g 50% aq. glutaraldehyde was added.
11. Heated to 20° C. and left to crosslink for 3 hours.

The microcapsules of the Comparative Sample 9 comprise a gelatin/acacia gum coacervate and polyisocyanate. The microcapsules are crosslinked using 0.04% glutaraldehyde at 20° C. for 3 hours.

### Microcapsule Stability

To measure the stability of the microcapsules, the microcapsules are dosed into a library base according to Table 3. The dosed bases were stored at either room temperature or at 40° C., and then dosed onto fabric.

TABLE 3
composition of Fabric Conditioner and Laundry Liquid

| Fabric Conditioner | | Laundry Liquid | |
|---|---|---|---|
| Component | Weight % | Component | Weight % |
| Quat surfactant | 11.3% | Anionic surfactants | 27% |
| Preservative | 0.03% | Nonionic surfactants | 12.5% |
| Calcium chloride | 0.1% | Propylene glycol | 12% |
| (10%) | | | |
| Microcapsule slurry | 0.02-0.5% | Preservative | 0.03% |
| Water | to 100% | Sequestrants | 2% |
| | | Opacifier | 1% |
| | | Microcapsule slurry | 0.2-0.5% |
| | | Water | to 100% |

At each of the time intervals (2, 4, 8 and 12 weeks, and also 1 week for Comparative Sample 8), the dosed bases were put onto a cloth, left to dry and rubbed.

The performance is rated 0 to 10, 10 being overpowering and 0 having no olfactory response. 12 weeks at 40° C. is equivalent to 3 years at RT and exceeds the shelf life of most products.

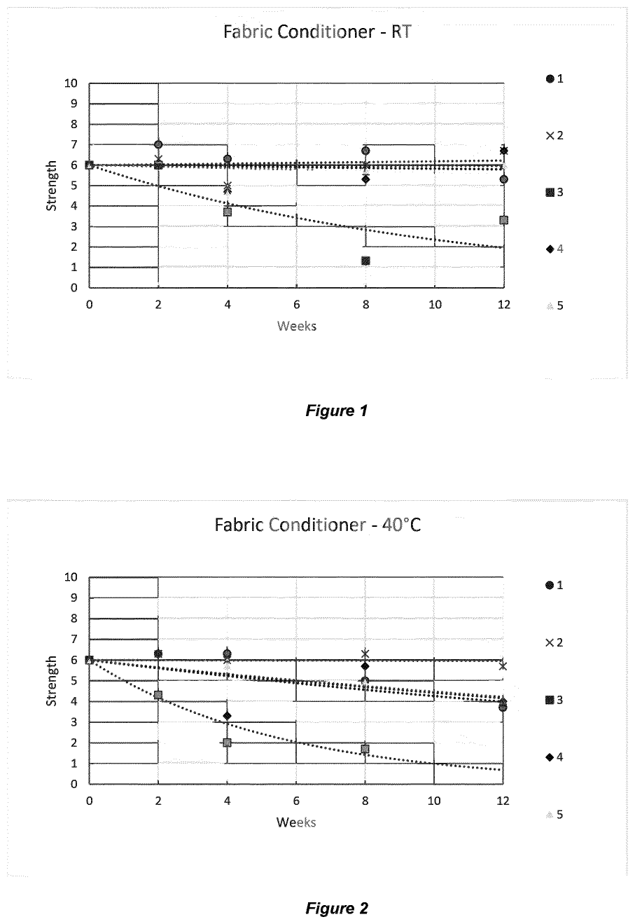

The stability and performance results are shown in FIGS. 1 to 12.

FIGS. 1 and 2 show the stability and performance results of the Samples 1 to 5 dosed into a fabric conditioner composition, at RT and at 40° C., respectively.

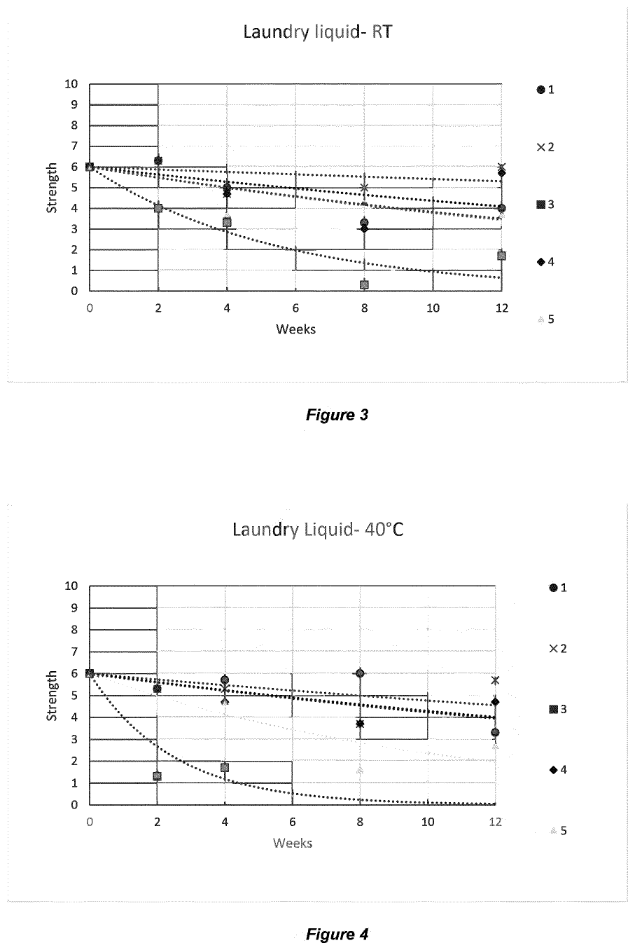

FIGS. 3 and 4 show the stability and performance results of the Samples 1 to 5 dosed into a liquid laundry composition, at RT and at 40° C., respectively.

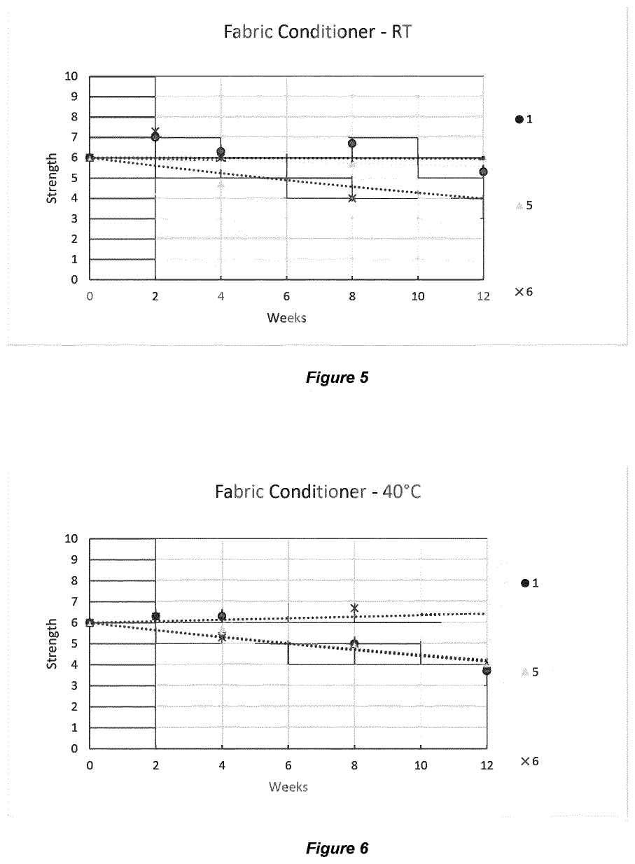

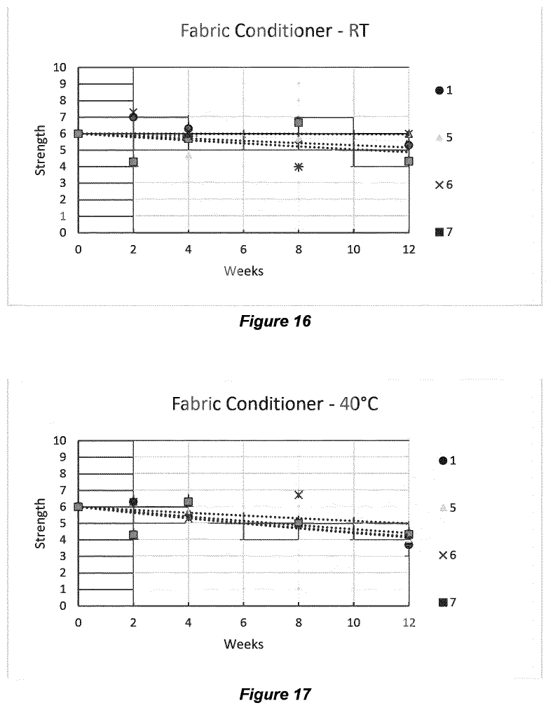

FIGS. 5 and 6 show the stability and performance results of the Samples 1, 5 and 6 dosed into a fabric conditioner composition, at RT and at 40° C., respectively. FIGS. 16 and 17 additionally show the same for Sample 7.

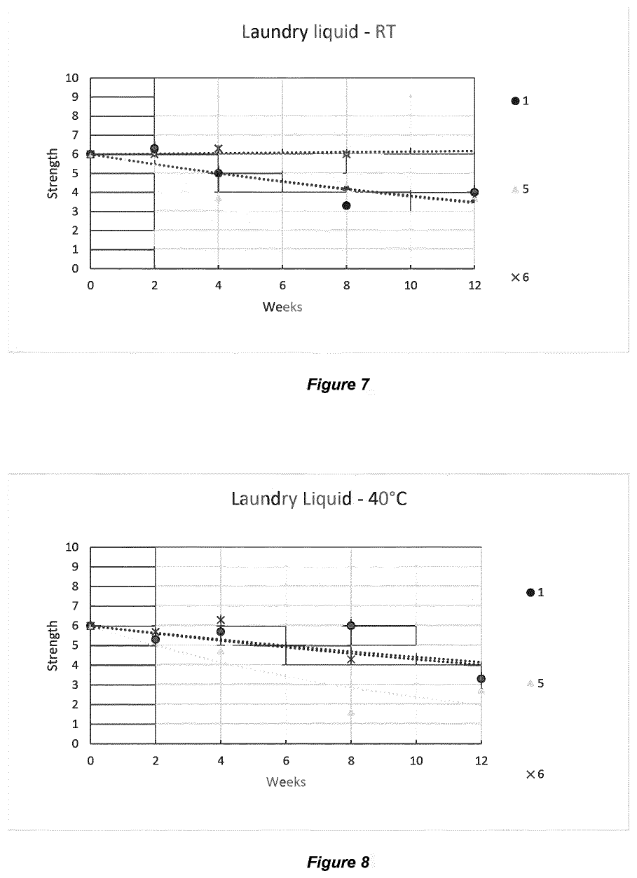

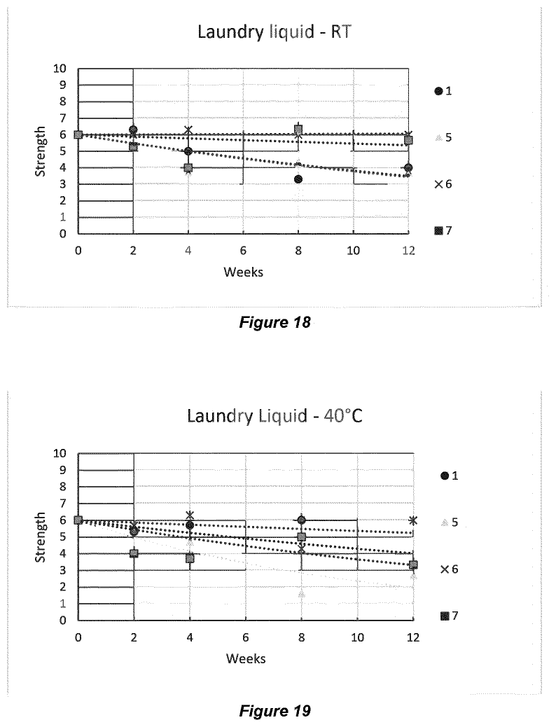

FIGS. 7 and 8 show the stability and performance results of the Samples 1, 5 and 6 dosed into a liquid laundry composition, at RT and at 40° C., respectively. FIGS. 18 and 19 additionally show the same for Sample 7.

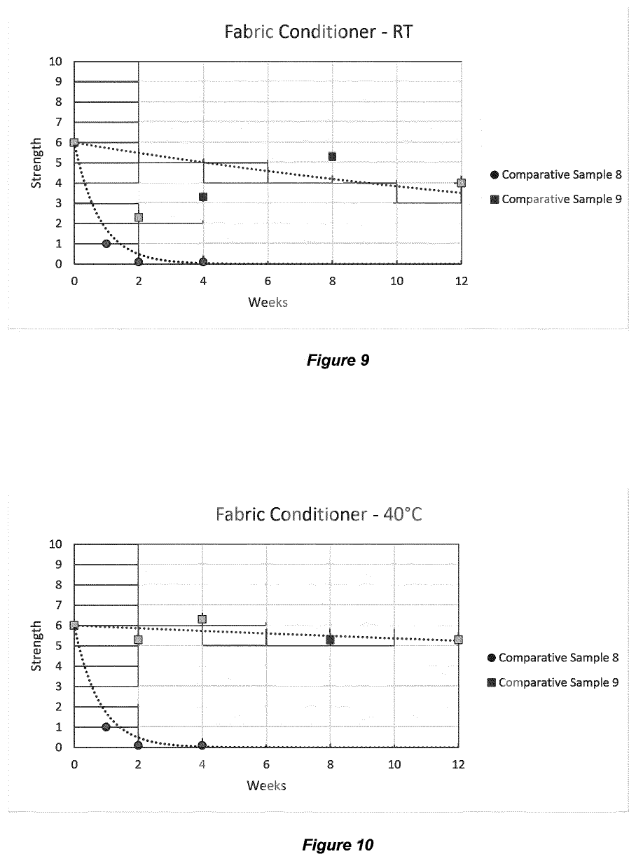

FIGS. 9 and 10 show the stability and performance results of Comparative Samples 8 and 9 dosed into a fabric conditioner composition, at RT and at 40° C., respectively.

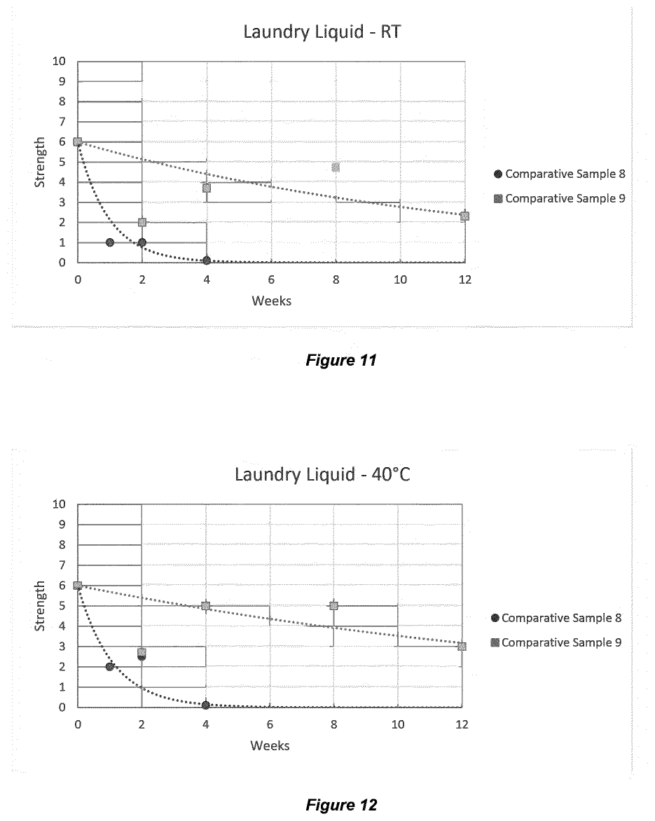

FIGS. 11 and 12 show the stability and performance results of the Comparative Samples 8 and 9 dosed into a liquid laundry composition, at RT and at 40° C., respectively.

All of the Samples 1 to 7 were stable at RT for 12 weeks in both the fabric conditioner and liquid laundry apart from Sample 3, which used a more aliphatic adduct isocyanate than the other compositions. For samples stored at 40° C. stability in fabric conditioner, the same results were seen and Sample 3 was the only one that failed. The stability in liquid laundry at 40° C. conforms with that of the fabric conditioner results apart for Sample 5. For Sample 5, the stability at 40° C. is slightly reduced compared to at RT, but the stability at both temperatures was still good.

Regardless of the type of chitosan used, the microcapsules were found to be stable with good performance.

One of the most important factors for the stability and performance is the choice of polyisocyanate. This can be seen by the poorer stability and performance of Sample 3, which uses a more aliphatic polyisocyanate.

Comparative Example 8 was produced according to the method in WO 2016/185171. The microcapsules include a coacervate using chitosan of vegan origin, and do not include a polyisocyanate.

The microcapsules of Comparative Example 9 include a coacervate of gelatin with acacia gum. The concentration of glutaraldehyde used is higher than the present invention, at 0.04 weight % of the dispersion.

The stability of Comparative Sample 8 is very poor and failed stability immediately at the 1-week interval regardless of the base formulation or the temperature it was stored at. It can be determined that complex coacervation capsules lacking the polyisocyanate are too poor of a barrier for protecting the fragrance inside from the base formulation.

For Comparative Sample 9, microcapsules stored at RT for weeks 2 and 4 did worse than microcapsules that were stored at 40° C. There is a drop in fragrance strength but overall, the stability results for these capsules was good in both bases. This may be attributed to the large amount of polyisocyanate in the microcapsules, which in turn may prevent the microcapsules from biodegrading.

In summary, like other complex coacervates, Comparative Sample 8 has poor stability in the base and is not suitable for applications that contain high levels of surfactants. Comparative Sample 9 is shown to have reduced stability when stored at room temperature compared to at 40° C.

### Microcapsule Laundrometer Performance

Performance testing was carried out using a Laundrometer (M228AA Launder-ometer, manufactured by SDL Atlas).

In preparation of the testing, cloths were washed at 90° C. with 100 g unperfumed laundry washing powder (Surcare non-bio laundry powder).

Each capsule formulation was dosed into concentrated fabric conditioner at 0.07% fragrance. Subsequently, 0.55 g of the conditioner was diluted into 399.45 g of cold water.

Each respective diluted fabric conditioner was placed into the canister with a cloth (100% cotton) and ten stainless steel balls. The Laundrometer was run for 15 minutes at 20° C. The cloths are quenched to remove excess water and left to dry for at least 24 hours. The cloths are assessed before and after rubbing.

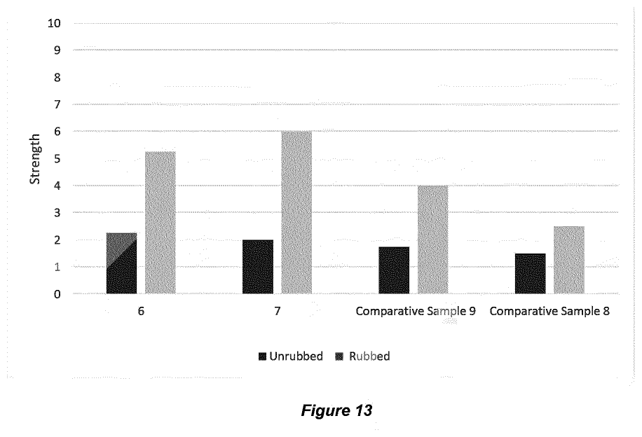

FIG. 13 illustrates the performance of various microcapsules of the current invention and the comparative examples. Towels were washed in a laundrometer at the same fragrance dosage and same amount of fabric conditioner.

The performance for both Sample 6 and Sample 7 was good. The difference between these two formulations is the process they are made (process 1 and process 2, respectively). There was no statistical difference between the performance of these two microcapsule samples. In comparison, the gelatin microcapsules of Comparative Sample 9 performed slightly worse, whilst the Comparative Sample 8 performed noticeably worse. The gelatine coacervate of Comparative Sample 9 is potentially a less adhesive coacervate and is therefore less likely to adhere to the substrate. The lack of polyisocyanate in Comparative Sample 8 leads to a shell that probably diffused fragrance during wash and drying.

### Additional Microcapsule Washing Machine Performance

Washing Machine Performance testing was carried out using a Miele Eco W Classic washing machine.

In preparation of the testing, towels were washed at 90° C. with 35 g unperfumed laundry washing powder (Surcare non-bio laundry powder). The washing machine drum was loaded with 5 cotton flannels and 2 bath towels.

Each capsule formulation was dosed into concentrated fabric conditioner at 0.07% fragrance. 30 mL of the dosed fabric conditioner was added to the fabric conditioner compartment of the machine drawer. 35 g of unperfumed laundry washing powder (Surcare non-bio laundry powder) was added to the drum.

The Washing machine was run for 1 hour 12 minutes at 40° C., 1,200 RPM spin. The towels and flannels are hanged to dry for at least 24 hours. The cloths are assessed before and after rubbing.

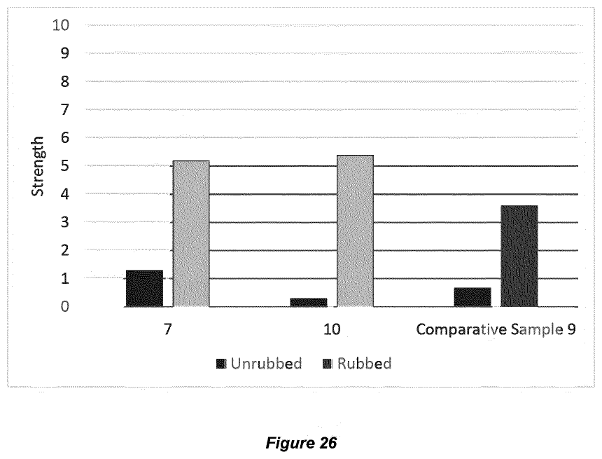

FIG. 26 illustrates the performance of various microcapsules of the current invention and the comparative examples.

The performance for both Sample 7 and Sample 10 was good. The difference between these two formulations is the amount of fragrance in the slurry (16% and 30%, respectively) with the level of isocyanate pro-rated. There was no statistical difference between the performance of these two microcapsule samples. In comparison, the gelatin microcapsules of Comparative Sample 9 performed worse. The gelatine coacervate of Comparative Sample 9 is potentially a less adhesive coacervate and is therefore less likely to adhere to the substrate.

### Biodegradability

Samples 1 to 5 were examined for biodegradability following the OECD 301F test. To ensure that the fragrance does not contribute to the biodegradation and produce a false positive, a non-degradable oil, ISO E Super (1-(2,3,8,8-tetramethyl-1,3,4,5,6,7-hexahydronaphthalen-2-yl) ethenone), was used as the inner phase in a control sample.

The OECD 301F test for ready biodegradability requires 60% theoretical oxygen demand (ThOD) by the end of a 10-day window; this window starts when biodegradation reaches 10% ThOD. Biodegradation beyond this 10-day window cannot be described as ready biodegradation but may be inherent biodegradable over a longer period. ECHA has identified that a minimum of 60% biodegradability in 60 days is required to combat the microplastic endemic.

### Moisture Content

All samples were tested for dry weight using a desiccator at room temperature for 48 hours. A desiccator was used instead of an oven due to potential volatility of the oil. Better precision in moisture content of replicates was observed for aliquots taken from vigorously shaken and stirred bottles. The samples, which were dried for longer, indicated moisture contents were more similar to the calculated water content (Table 4).

TABLE 4
Summary of mean density, and mass loss from
samples compared to calculated water content

| | | Mean | | Mean mass | Calculated |
|---|---|---|---|---|---|
| | | density/ | | loss on | Water |
| Sample | Replicates | g/mL | | drying % | Content % |
| 1 | 6 | 1.021 | (0.004) | 77.30 (0.06) | 78.36 |
| 3 | 4 | 1.018 | (0.008) | 76.25 (0.04) | 78.32 |
| 4 | 6 | 1.027 | (0.008) | 78.73 (0.04) | 78.66 |
| 5 | 6 | 1.032 | (0.006) | 79.45 (0.03) | 78.29 |
| Oil (ISO E Super) | 4 | 0.969 | (0.01) | 0.224 (0.07) | — |

Sample 3 was dried for 21 hours and all other samples were dried for 48 hours.

The values in parentheses are the standard deviation values.

### Biodegradability Test

The OECD test requires a number of criteria to be met for consideration of a valid test, which were satisfied under the conditions used here.

Three different mixing regimes were investigated to facilitate dispersal of the capsule material and oil in the Oxitop bottles. Different types of stir-bars were trialled; a regular octahedral pivot ring; a triangular/wedge shape; and cross-shaped. Batches of six bottles each containing 3×oil; 1 blank; 1 reference compound; and 1 sample (3) were placed on three magnetic stirrers on settings which reflected near the maximum speed at which the stir-bars remained coupled-slowest for the wedge, and similar speeds for the other two with the cross-shape generating a deeper vortex potentially dispersing the oil better.

The slow wedge stir-bars yielded the highest biological oxygen demand (BOD) values (mg/L) of the triplicates in all samples (Table 5). It is plausible that the slower mixing regime, allowed for better growth and structure of the microbial flocs thereby facilitating biodegradation.

TABLE 5
Maximum BOD (mg/L) using three different magnetic stir-bars

| | Blank | Reference | Sample 3 | Oil |
|---|---|---|---|---|
| Stir-bar | BOD | BOD | BOD | BOD |
| type | (mg/L) | (mg/L) | (mg/L) | (mg/L) |
| Wedge | 22.3 | 85.4 | 52.5 | 16.6 |
| Cross | 17.1 | 76.2 | 44.7 | 13.1 |
| Octahedral | 17.1 | 78.8 | 40.7 | 14.9 |

In preliminary tests, it was found that the capsule slurry can be pipetted directly from a vigorously shaken, rapidly stirred, bulk sample in suitable quantity to give sufficiently accurate and representative replicate sub-samples.

Test samples can be weighed and transferred into the test bottle by rinsing with water from a wash bottle without leaving any residue on plastic surfaces of weigh boats.

The oil may affect the test in two ways. First, by being slightly biodegradable, it may contribute to the overall oxygen demand. Second, it may interfere with the oxygen exchange at the water/air boundary when released from capsules. The effect of biodegradability was found to be very small, or smaller than the interference effect, in the test conditions used. Scoping tests indicated that degradation of the oil used in tests was negligible under the experimental conditions, with a small inhibitory effect. Mean maximum oil BOD (n=9) was 14.9±1.7 mg/L compared to inoculum blanks (n=3) of 18.8±3.0 mg/L. A separate oil treatment can account for the degradation of oil in the same inoculum. Furthermore, a treatment using the same amount of oil can be used to assess the effect on biodegradation of a reference sample.

A target concentration of 100 mg/L capsule components was calculated to yield ThOD somewhat higher than the recommended OECD standard, however, a scoping test on Sample 3 demonstrated maximum mean degradation of 29%, thus the higher value was considered acceptable to prevent the capsule material signal being potentially swamped by other test components. Simultaneously, a target concentration for oil was set at 152 mg/bottle of oil to represent the mean oil proportion in the bulk samples, thus any contributory effect of the oil component (if observed) could be directly subtracted from the whole samples.

Using the samples 1 to 5 and Oil (ISO E Super), ThOD was calculated and reported in Table 6. The capsule material was calculated separately from the water and oil content. The whole sample weight required to contain the correct amount of capsule material (100 mg/L) was calculated.

TABLE 6
Sample physio-chemical properties (means of 4 or
6 replicates) and ThOD (without nitrification)

| | | | | ThOD | ThOD | |
|---|---|---|---|---|---|---|
| | | | | (capsule) | (capsule) | Conc. |
| | | Calc. | Calc. | mg | mg | Sample |
| | Mean | water | Oil | O₂/mg | O₂/mg | in test |
| | density | content | content | capsule | whole | soln. |
| Sample | g/mL | % | % | only | sample | mg/L |
| 1 | 1.021 | 78.36 | 16.15 | 1.26 | 0.069 | 1817 |
| 3 | 1.018 | 78.32 | 16.14 | 1.28 | 0.071 | 1803 |
| 4 | 1.027 | 78.66 | 16.24 | 1.23 | 0.063 | 1960 |
| 5 | 1.032 | 78.29 | 16.41 | 1.24 | 0.066 | 1885 |
| Oil (ISO E | 0.969 | — | — | 3.00 | — | 306 |
| Super) | | | | | | |

Activated sludge was collected from the aeration tank of Cotton Valley wastewater treatment plant receiving predominantly domestic sewage from the Milton Keynes area.

Approximately 3 L of activated sludge was collected. On receipt in the lab, the sample was sieved to 250 μm to reduce the suspended solids concentration. The filtrate was preconditioned by aeration at 800 ml/min at 22° C. for 7 days.

Additionally, the suspended solids concentration is identified by calculating the dry weight of the inoculum and identified to be 29 mg/L.

Sodium acetate was used as reference material.

### Test Conditions

Test duration was initially 28 days and later extended to 60 days to verify inherent biodegradability. Temperature was controlled at 22° C.

The test subject was taken to be the capsule material only. Preliminary tests showed minimal biodegradation of the oil component in the conditions of the test; however, additional treatments were added to account for the effect of biodegradation of oil alone, and any toxic effect of the oil (Table 7). The total volume in the test flask was 496 mL, including the water content of the sample.

TABLE 7
Treatment list of test samples

| | No. | |
|---|---|---|
| Treatment | replicates | Note |
| Inoculum only | 2 | Blank |
| Reference compound + inoculum | 1 | Positive control |
| Sample of oil only + inoculum | 2 | Contribution of oil |
| | | alone |
| Sample 1 + inoculum | 2 | Standard test |
| Sample 3 + inoculum | 2 | Standard test |
| Sample 4 + inoculum | 2 | Standard test |
| Sample 5 + inoculum | 2 | Standard test |
| Sample 1 + reference + inoculum | 1 | Toxicity control |
| Sample 3 + reference + inoculum | 1 | Toxicity control |
| Sample 4 + reference + inoculum | 1 | Toxicity control |
| Sample 5 + reference + inoculum | 1 | Toxicity control |
| Sample of oil + reference + inoculum | 1 | Toxicity effect of oil |

### Results

Results for biodegradability as percentage of ThOD are shown in Table 8. The results for the oil at the end of the 10-day window is not reported, as it did not reach the 10% ThOD level that starts the 10-day window. The biodegradability at 28 days, which can be taken as plateau level, and at 60 days, are also shown. The lag, taken as the time to reach 10% ThOD, is also reported.

TABLE 8
Biodegradability results of samples

| | | Biodegrad- | | |
|---|---|---|---|---|
| | | ability, | Biodegrad- | Biodegrad- |
| | | end 10-day | ability | ability |
| | Lag | window | at 28 days | at 60 days |
| Sample | (days) | (% ThOD) | (% ThOD) | (% ThOD) |
| 1 | 5.44 | 31.3 | 39.1 | 43.8 |
| 3 | 5.60 | 32.2 | 38.9 | 45.5 |
| 4 | 5.60 | 36.8 | 42.1 | 46.9 |
| 5 | 4.98 | 52.0 | 63.2 | 70.1 |
| Oil | N/A | N/A | 0.15 | 1.0 |
| (ISO E | | | | |
| Super) | | | | |

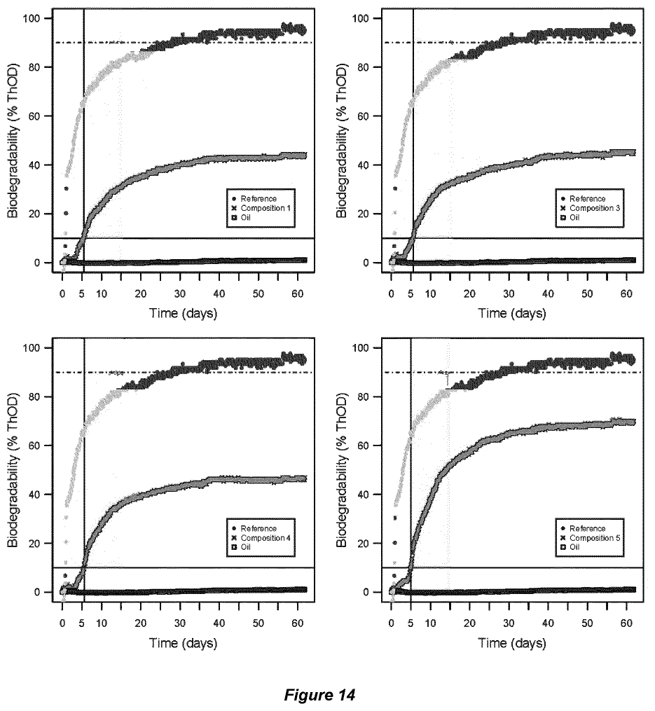

The biodegradation percentage is illustrated for each sample in FIG. 14. The degradation phase, defined as the period from the end of lag to 90% ThOD, is not shown, since the samples did not reach this level.

Samples 1, 3 and 4 all performed similarly and showed some level of biodegradability but below 60% in 28 days. Sample 5 was the most biodegradable out of the samples, degrading to 63% after 28 days and to 70% in 60 days. Sample 5 reached the threshold of 60% after 28 days and is consequently inherently biodegradable.

The most defining property for the biodegradability wasn't the level of isocyanate or the type of polyisocyanate but the amount of glutaraldehyde that was added. Only the sample with a low glutaraldehyde content according to the present invention, Sample 5, was found to be biodegradable.

Interestingly, the aromaticity of the polyisocyanate used and the amount of polyisocyanate had little influence on the biodegradability. The more aliphatic Sample 3 and the lower polyisocyanate Sample 4 performed very similar to Sample 1.

### Validity

Validity requirements according to OECD 301F are shown in Tables 9 and 10.

TABLE 9
Validity criteria for test run

| Validity Criteria | Result |
|---|---|
| Percentage degradation of the reference compound has reached | Pass |
| the pass level (60% ThOD within a 10- day window) by day 14 | |
| The Oxygen uptake of the inoculum blank not greater than 60 | Pass |
| mg/l in 28 days (normally 20-30 mg O₂/L) | |
| pH at end of test | n.d.* |
| Difference between replicates <20%, end of 10-day window | Pass |
| Difference between replicates <20%, end of 28-day test | Pass |
| Difference between replicates <20%, end of 60-day test | Pass |
| Sample toxicity tests, containing both the test substance and a | Pass |
| reference compound, above 25% (based on total ThOD) within | |
| 14 days | |

*n.d. = not determined. pH at the end of test was not measured due to leakage of NaOH on removal of the CO₂ trap.

TABLE 10
Sample specific validity criteria

| | Difference between | Difference between | Toxicity test, |
|---|---|---|---|
| | Replicates, % ThOD | Replicates, % ThOD | % total ThOD |
| | at lag + 10 days | at 60 days | at 14 days |
| Sample | (<20%) | (<20%) | (>25%) |
| 1 | 4.2 | 3.1 | 49.8 |
| 3 | 5.1 | 4.0 | 48.7 |
| 4 | 5.4 | 3.2 | 50.0 |
| 5 | 6.4 | 3.2 | 61.5 |

Toxicity tests were run, containing both sample and reference material. These passed the validity requirements based on the ThOD of sample and reference material. These were calculated without the inclusion of the oil content, known to be of low biodegradability. It was not possible to allocate the biodegradation to specific components of the test mixture. However, if it is assumed the sample to the same degree as in sample test mixtures, this represents a small suppression of biodegradability of the reference material in each case. This may simply be due to the additional material approaching the maximum capacity of the test chambers, in terms of absorption of oxygen or maintaining pressure.

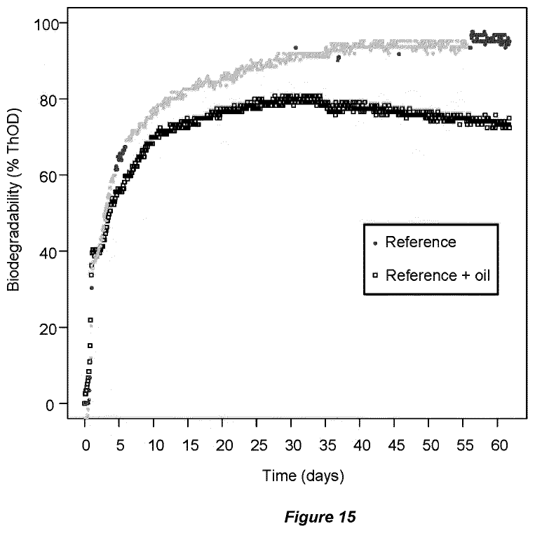

Toxicity tests using a quantity of oil matching that in the samples could not be calculated in the same way as for samples, due to very high ThOD loading for the poorly biodegradable oil. Instead the quantity of oil was approximately matched to the oil content of samples and treated as an interference test. The oil could either degrade and add to the biodegradability signal or suppress the degradation of the reference material. As an indication, the oil only treatment was subtracted from the reference plus oil results (FIG. 15). Based on biodegradation of reference material only, the oil plus reference treatment reached 71.6% ThOD at 14 days, about 10% lower than the reference material alone (81.7% ThOD). Unlike the reference plus sample toxicity tests, it is unlikely this was due to approaching the limits of the test equipment, since biodegradability of the oil is very small. It is therefore possible that microcapsules could show suppressed biodegradability due to the presence of oil.

In summary, the type of polyisocyanate and concentration of polyisocyanate used has little to no effect on the biodegradability. Samples 3 and 4 achieved 38.9% and 42.1% in 28 days, respectively. The concentration of the crosslinking agent is the most influential factor in biodegradability. Sample 5 with a lower glutaraldehyde content achieved 63.2% in 28 days, classifying for inherent biodegradability.

### Additional Results and Discussion

### Varying Glutaraldehyde Content

### Capsule Preparation

Samples A, B and C were prepared according to process 2 and Sample D was prepared according to process 3. The compositions of the microcapsule samples are detailed in Table 11.

Samples B, C and D were examined for biodegradability following the OECD 301F test. The biodegradability testing method is as described above.

Sample B and C were made using less shell material, less isocyanate and a larger amount of glutaraldehyde than Sample 5.

Sample C is the same composition as described in WO 2021/018947 at Protocol 5, Example 1 and Table 4. Sample C has a higher glutaraldehyde content compared to Samples B and D, for example and also compared to Samples 5, 6 and 7.

Sample D was made using chitosan and isosorbide as the coacervate material. Due to the charge difference between isosorbide and acacia gum a different pH compared to Samples B and C, for example, was required to reach the isoelectric point of coacervation.

TABLE 11
Summary of microcapsule compositions.

| | | | Acacia | | | | | | | | | |
|---|---|---|---|---|---|---|---|---|---|---|---|---|
| | Chitosan | | Gum² | | | | | Fragrance⁴ | Polyisocyanate⁵ | | | |
| | Kitozyme | | Type 8074 | | 20% | 5% | | ISO E | Takenate | | | |
| Sample | CsH¹ | | RD | D-Isosorbide³ | HCl | TEA | | SUPER | D-110N | Glutaraldehyde | | Water |
| A | 1.5 | g | 10 g | — | 4 g | 19.25 | g | 112 g | 2.88 g | 0.0675 | g | 500 g |
| B | 1.5 | g | 10 g | — | 4 g | 19.25 | g | 112 g | 2.88 g | 0.135 | g | 500 g |
| C | 1.5 | g | 10 g | — | 4 g | 19.25 | g | 112 g | 2.88 g | 0.27 | g | 500 g |
| D | 2 | g | — | 1 g | 4 g | 82.5 | g | 112 g | 2.88 g | 0.05 | g | 500 g |

¹KIOSMETINE-CSH, sourced from KitoZyme is derived from Agaricus bisporus. Degree of acetylation: 0-30% (mol %), viscosity: 12-25 cP
²Type 8074 RD, sourced from Norevo GmbH
³D-Isosorbide 98%, sourced from Alfa Aesa
⁴ISO E Super (1-(2,3,8,8-tetramethyl-1,3,4,5,6,7-hexahydronaphthalen-2-yl)ethenone), sourced from Eternis Fine Chemicals UK Limited
⁵75% solution in ethyl acetate, sourced from Mitsui Chemicals

### Biodegradability

Using the specific compositions, ThOD was calculated and reported in Table 12. The capsule materials were calculated separately from the water and oil content. The whole sample weight required to contain the correct amount of capsule material (100 mg/L) was calculated.

TABLE 12
Sample physio-chemical properties (means of 4 or
6 replicates) and ThOD (without nitrification).

| | | | ThOD | ThOD | |
|---|---|---|---|---|---|
| | | | (capsule) | (capsule) | Conc. |
| | Calc. water | Calc. oil | mg O₂/mg | mg O₂/mg | sample in |
| | content | content | capsule | whole | test soln. |
| Sample | % | % | only | sample | mg/L |
| B | 80.57 | 16.67 | 1.26 | 0.032 | 1926 |
| C | 80.28 | 16.69 | 1.26 | 0.034 | 1855 |
| D | 82.48 | 15.92 | 1.43 | 0.022 | 3226 |

Activated sludge was collected from the aeration tank of Cotton Valley wastewater treatment plant receiving predominantly domestic sewage from the Milton Keynes area.

Approximately 3 L of activated sludge was collected. On receipt in the laboratory, the sample was sieved to 250 μm to reduce the suspended solids concentration. The filtrate was pre-conditioned by aeration at 800 mL/min at 22° C. for 5 days.

The suspended solids concentration is identified by calculating the dry weight of the inoculum and identified to be 29 mg/L.

Sodium acetate was used as reference material.

### Test Conditions

Test duration was initially 28 days and later extended to 33 days to verify inherent biodegradability. The temperature was controlled at 22° C.

The capsule material was used as the test subject. Preliminary tests showed minimal biodegradation of the oil component in the conditions of the test; however, additional treatments were added to account for the effect of biodegradation of oil alone, and any toxic effect of the oil (Table 13). The total volume in the test flask was 496 mL, including the water content of the sample.

TABLE 13
Treatment list of test samples

| | No. | |
|---|---|---|
| Treatment | replicates | Note |
| Sample B + inoculum | 2 | Standard test |
| Sample C + inoculum | 2 | Standard test |
| Sample D + inoculum | 2 | Standard test |
| Inoculum only | 1 | Blank |
| Reference | 1 | Positive control |
| Sample of oil | 1 | Toxicity effect of oil |
| Sample of oil + reference | 1 | Toxicity effect of oil |
| Sample of oil (reduced) + reference | 1 | Toxicity effect of oil |

### Results

Results for biodegradability as a percentage of ThOD are shown in Table 14. The results for the oil at the end of the 10-day window is not shown, as it did not reach the 10% ThOD level that starts the 10-day window. The biodegradability at 28 days, which can be taken as plateau level, is also shown. The lag, taken as the time to reach 10% ThOD, is also reported.

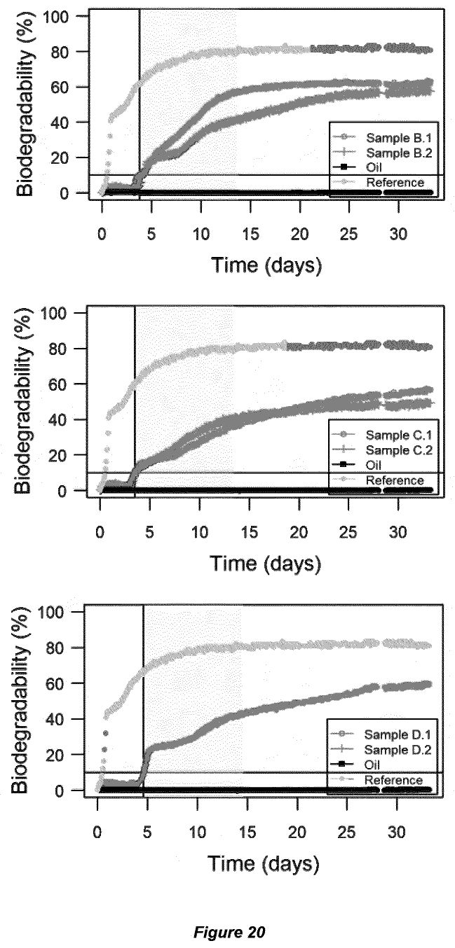

The biodegradation percentage is illustrated for each sample in FIG. 20. The degradation phase, defined as the period from the end of lag to 90% ThOD, is not shown, since the tested samples did not reach this level.

Sample B performed slightly better than Sample C and was slightly below the 60% biodegradability in 28 days achieved by Sample 5. The concentration of coacervate crosslinker appears to have a significant effect on the biodegradability.

Sample D made using different polymers appears to have performed as well as Sample B although the ratio of coacervate to crosslinker was less. This is probably due to isosorbide having a much smaller, simple chemical structure than acacia gum, making it more readily available for biodegradation.

TABLE 14
Biodegradability results of samples

| | | Biodegradability, | Biodegradability |
|---|---|---|---|
| | Lag | end 10-day window | at 28 days |
| Sample | (days) | (% ThOD) | (% ThOD) |
| B | 4.12 | 48.6 | 58.1 |
| C | 3.58 | 38.0 | 50.6 |
| D | 4.67 | 42.5 | 57.6 |
| Oil | N/A | N/A | −0.1 |
| (ISO E | | | |
| SUPER) | | | |

### Validity

Validity requirements according to OECD 301 are shown in Table 15 with further details per sample in Table 16.

Toxicity tests were not carried out on the samples. Previous testing showed that the samples with much larger quantities of polymer, crosslinker and isocyanate were non-toxic, and it is therefore assumed that these samples would also be non-toxic.

TABLE 15
Validity criteria for test run

| Validity Criteria | Result |
|---|---|
| Percentage degradation of the reference compound has reached | Pass |
| the pass level (60% ThOD) by day 14 | |
| The Oxygen uptake of the inoculum blank not greater than 60 | Pass |
| mg/L in 28 days (normally 20-30 mg O₂/L) | |
| pH at end of test | Pass |
| Difference between replicates <20%, end of 10-day window | Pass |
| Difference between replicates <20%, end of 28-day test | Pass |
| Sample toxicity tests, containing both the test substance and a | N/A |
| reference compound, above 25% (based on total ThOD) within | |
| 14 days | |

*the reference compound was readily biodegradable, reaching 60% ThOD (71.9%) in the 10-day window.

TABLE 16
Sample specific validity criteria

| | Difference between replicates | Difference between replicates |
|---|---|---|
| | % ThOD at lag + 10 days | % ThOD at 28 days |
| Sample | (<20%) | (<20%) |
| B | 16.7 | 7.2 |
| C | 3.1 | 3.1 |
| D | 5.0 | 5.4 |

### Further Analysis

By calculating the ratio of glutaraldehyde to the amount of coacervate polymer present, it is possible to plot the level of biodegradability against the amount of glutaraldehyde to further illustrate the importance of the ratio of crosslinker to coacervate.

Table 17 details the amount of coacervate and glutaraldehyde used, the ratio and the biodegradability achieved for that sample.

TABLE 17
Coacervate and glutaraldehyde amounts for the different
compositions measured for biodegradability

| | | | Ratio | | Biodegrad- |
|---|---|---|---|---|---|
| | Total | Glutar- | Coacer- | | ability |
| | Coacervate | aldehyde | vate:Glutar- | Log | at 28 days |
| Sample | (g) | (g) | aldehyde | (Ratio) | (% ThOD) |
| 1 | 27.6 | 1.5 | 18.4 | 1.26 | 39.1 |
| 3 | 27.6 | 1.5 | 18.4 | 1.26 | 38.9 |
| 4 | 27.6 | 1.5 | 18.4 | 1.26 | 42.1 |
| 5 | 27.6 | 0.075 | 368 | 2.57 | 63.2 |
| B | 11.5 | 0.135 | 85.2 | 1.93 | 58.1 |
| C | 11.5 | 0.27 | 42.6 | 1.63 | 50.6 |

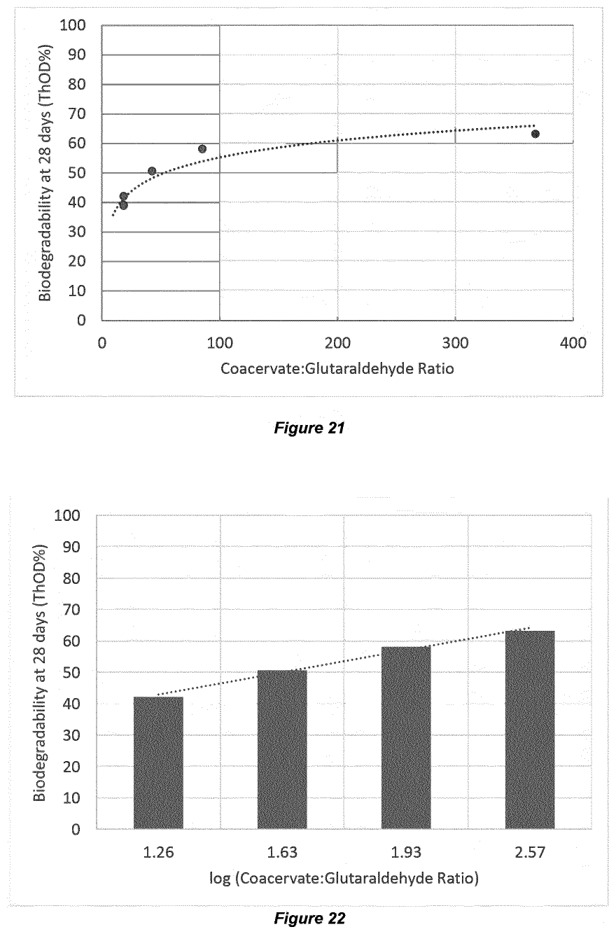

FIGS. 21 and 22 show the plotted values of Table 17. An increase of coacervate to glutaraldehyde ratio leads to a more biodegradable material. The significance of the biodegradability is found to be logarithmic. From these results, it can be seen that increasing the level of glutaraldehyde has a detrimental alteration to the biodegradability.

In summary, the concentration of coacervate crosslinker was found to be an influential factor in biodegradability and demonstrated to have a significant effect on the biodegradability.

### IR Analysis of Microcapsules

These experiments were carried out to investigate the effects of the crosslinker content on the structure of the microcapsules that are produced.

Infrared spectrometry can provide information of compounds that are a unique expression of the structure. The molecules bond vibrations resonate at certain absorption frequencies which superimpose and are displayed in the spectrum.

Glutaraldehyde is a covalent crosslinker. The level of crosslinking was hypothesized to increase with increasing in concentration, and this was expected to be reflected in the IR spectra. This set of experiments thus examine how the concentration of glutaraldehyde used to prepare microcapsules alters the IR spectra.

Glutaraldehyde is introduced to crosslink the coacervate wall formed during the manufacturing process of the microencapsulated fragrances. The microcapsules evaluated here comprise a chitosan and gum Arabic coacervate with varying amounts of glutaraldehyde crosslinker. Samples A, B and C as detailed in Table 11 were tested. Sample A has the lowest glutaraldehyde content, whilst Sample C has the highest.

### Capsule Preparation

The microcapsule shells were prepared for IR evaluation using the following method:
1. Microcapsule slurry was centrifuged at 2800 RPM for 10 minutes.
2. The microcapsule layer was decanted and left to dry overnight.
3. 5 g of the dried capsules was broken using a pestle and mortar.
4. The crushed capsules were washed with 5 g hexane.
5. 2 g DI water was added to the mixture.
6. The mixture was further centrifuged at 2800 RPM for 10 minutes.
7. The hexane and water was removed and the crushed capsules were collected.
8. The crushed capsules were further washed with ethanol and left to dry.

### Infrared Spectra

IR spectra was performed using a Shimadzu IR Spirit-T spectrophotometer with DLATGS detector equipped with a diamond QATR-S. Using the Happ-Genzel model, the resolution and the number of scans were 2 cm-1 and 45, respectively.

### Infrared Results

During the microencapsulation process coacervation can be induced, for example, by changing the pH to the isoelectric point. At the isoelectric point, amines groups present in chitosan become positively charged and hydroxy moieties present in acacia gum become negatively charged.

Without wishing to be bound by theory, it is believed that glutaraldehyde can react with either amines or alcohols to crosslink the polymers. The amines in the chitosan and the alcohols in the acacia gum are believed to be available for crosslinking. Three proposed crosslinking mechanisms are illustrated in Schemes 1 to 3. These are believed to be the main mechanisms, however glutaraldehyde might react with the other alcohols present in the chitosan and acacia gum. As the level of glutaraldehyde increases, the amines and alcohols are expected to react and thus lead to an increase in ether and imine bonds.

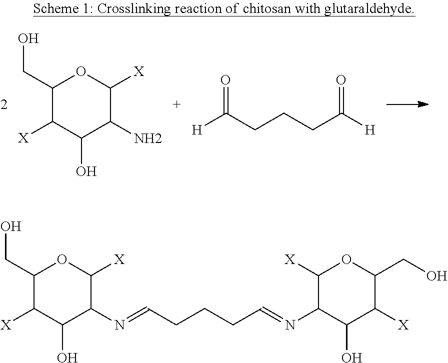

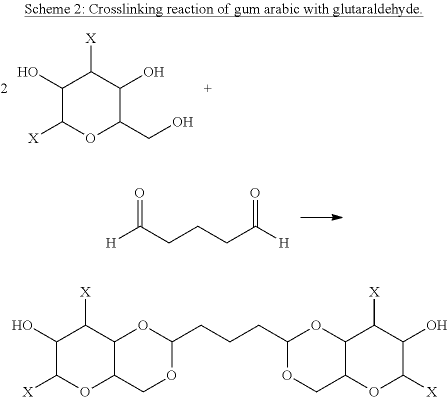

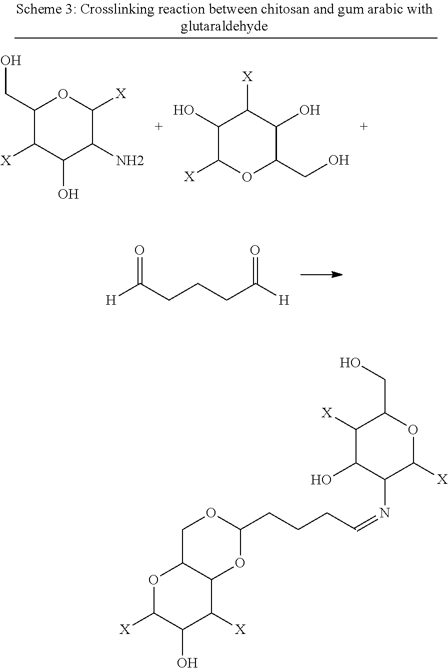

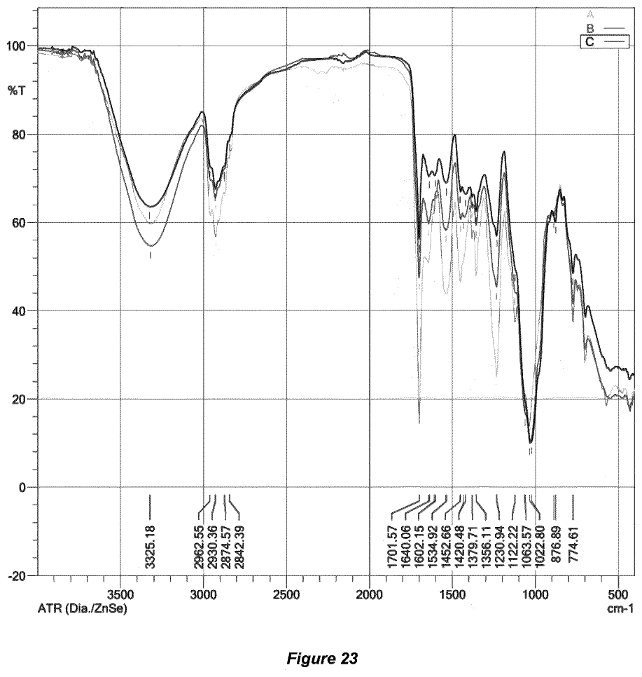

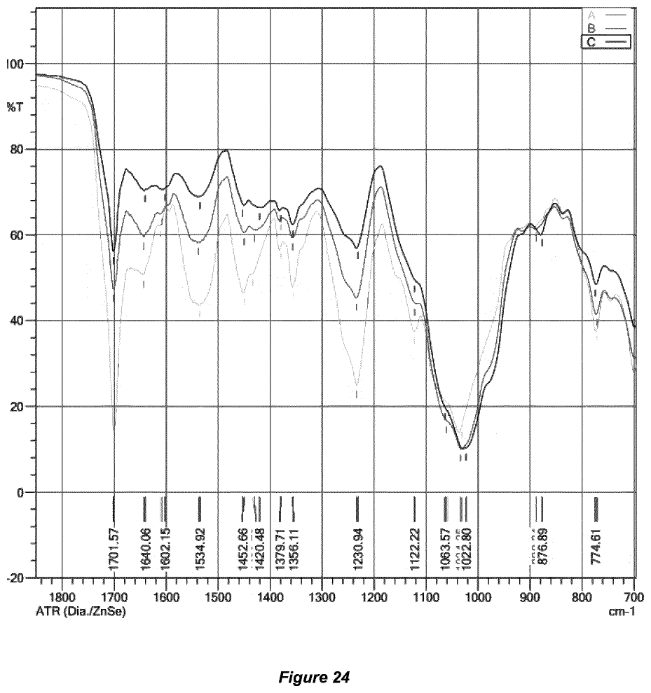

FIG. 23 illustrates the spectra of Samples A, B and C at the frequency range 400 to 4,000 cm⁻¹, and FIG. 24 illustrates the same in the frequency range 700 to 1,850 cm⁻¹. The absorption bands are labelled in Table 18.

There is a clear transition in the absorption of the microcapsule samples as the level of glutaraldehyde changes. Most peaks are less intense with increased glutaraldehyde with the exception for the peaks at 1,020-1,120 cm⁻¹ and at 1,600 cm⁻¹ which correspond to C—O stretch (ether) and C═N stretch, respectively. There is a decrease in absorption for N—H bending, and the two C—OH bending. These changes agree with the equations above and indicates increased level of crosslinking with increasing amount of glutaraldehyde used.

TABLE 18
IR absorption frequencies and their corresponding vibrational mode

| Wavenumber (cm⁻¹) | Vibrations |
|---|---|
| 775 | Sp² C—H δ (Aromatic) |
| 875 | Sp² C—H δ (alkene) |
| 1020-1120 | C—O u (Ether) |
| 1230 | C—O u (Ester) |
| 1330-1390 | sp³ C—H δ |
| 1420 | CH₂—OH δ |
| 1450 | CH—OH δ |
| 1535 | N—H δ |
| 1600 | C═N u |
| 1640 | C═O u (Amide) |
| 1700 | C═O u |

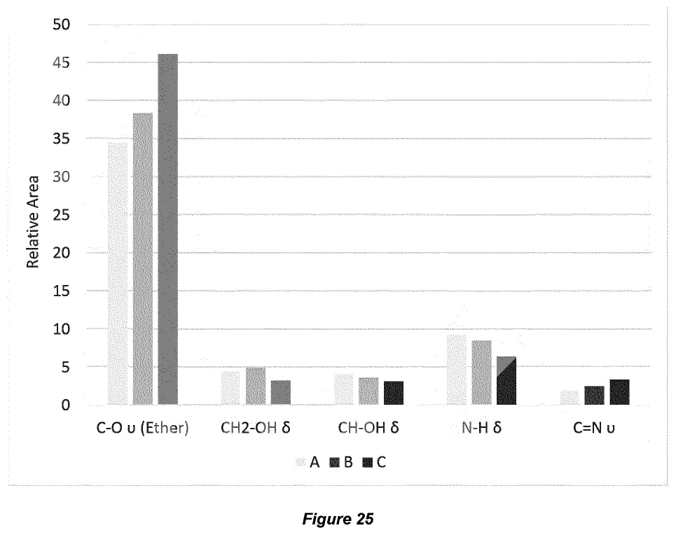

To identify the change between the microcapsule samples, the area relative to each peak was calculated. The relative area for the crosslinking peaks is detailed in Table 19 and illustrated in FIG. 25.

The C—O ether stretch vibration notably increases with glutaraldehyde concentration, whilst the alcohol vibrations decrease. The large increase in the ether vibration indicates the acetal formation and the crosslinking for the acacia gum. As the amine bend vibration decreases, the imine stretch vibration increases indicating the crosslinking for the chitosan.

TABLE 19
IR relative area for compositions A, B & C.

| | Wavenumber | Relative Area | | |
|---|---|---|---|---|
| Vibrations | (cm⁻¹) | A | B | C |
| C—O u (Ether) | 1020-1120 | 34.55 | 38.36 | 46.09 |
| CH2—OH δ | 1535 | 4.48 | 4.91 | 3.22 |
| CH—OH δ | 1600 | 4.06 | 3.65 | 3.15 |
| N—H δ | 1535 | 9.34 | 8.50 | 6.38 |
| C═N u | 1600 | 1.98 | 2.54 | 3.41 |

From these IR results, it can be concluded that glutaraldehyde in the samples is crosslinking the coacervate polymers present in the microcapsules. As the level of glutaraldehyde increases, the level of crosslinking also increases.

### REFERENCES

A number of publications are cited above in order to more fully describe and disclose the invention and the state of the art to which the invention pertains. Full citations for these references are provided below. The entirety of each of these references is incorporated herein.
US 2015/250689
US 2005/0112152
US 2020/0281826
WO 2019/096817
WO 2021/018947
CN 106614564
US 2011/0111020
US 2020/0164332
WO 2020/131866
WO 2020/131890
WO 2016/185171

1. A method for producing a microcapsule, comprising the steps of:
(i) providing a complex coacervate of a polycation with a polyanion in an aqueous phase;
(ii) dispersing an organic phase comprising a polyisocyanate in the aqueous phase;
(iii) inducing encapsulation of the organic phase by the complex coacervate and polymerising the polyisocyanate at the interface of the organic phase; and
(iv) adding a crosslinking agent to the dispersion of step (iii) in an amount of up to 0.03% by weight of the dispersion and permitting the crosslinking agent to form crosslinks.

2. The method according to claim 1, wherein the crosslinking agent is added in an amount of 0.001% to 0.03% by weight of the dispersion of step (iii).

3. A method for producing a microcapsule, comprising the steps of:
(i) providing a complex coacervate of a polycation with a polyanion in an aqueous phase;
(ii) dispersing an organic phase comprising a polyisocyanate in the aqueous phase;
(iii) inducing encapsulation of the organic phase by the complex coacervate and polymerising the polyisocyanate at the interface of the organic phase; and
(iv) adding a crosslinking agent to the dispersion of step (iii) in an amount of up to 10 parts by weight relative to 1,000 parts by weight of the complex coacervate provided in step (i), and permitting the crosslinking agent to form crosslinks.

4. The method according to any one of claims 1 to 3, wherein step (iv) comprises a stage of permitting the crosslinking agent to form crosslinks at a temperature of 30° C. or more.

5. The method according to claim 4, wherein the temperature is from 30° C. to 50° C.

6. The method according to any one of claims 1 to 5, wherein the polycation is selected from a polysaccharide, a plant protein and a milk protein.

7. The method according to any one of claims 1 to 6, wherein the polycation is a polysaccharide.

8. The method according to any one of claims 1 to 7, wherein the polycation is chitosan.

9. The method according to any one of claims 1 to 8, wherein the polyanion is selected from cellulose, starch, modified starch, agar, alginate, xanthan gum, plant gum, casein, zein, agarose, polyvinyl alcohol, isosorbide, derivatives thereof and salts thereof.

10. The method according to any one of claims 1 to 9, wherein the polyanion is acacia gum.

11. The method according to any one of claims 1 to 10, wherein the crosslinking agent is glutaraldehyde.

12. The method according to any one of claims 1 to 11, wherein step (i) comprises forming the complex coacervate.

13. The method according to any one of claims 1 to 12, wherein step (ii) comprises dispersing the organic phase to form droplets of the organic phase in the aqueous phase.

14. The method according to claim 13, wherein step (iii) comprises encapsulating the droplets of the organic phase by the complex coacervate.

15. The method according to any one of claims 1 to 14, wherein the polyisocyanate comprises an aromatic group.

16. The method according to any one of claims 1 to 14, where the polyisocyanate comprises one or more selected from xylylene diisocyanate (XDI), hydrogenated xylylene diisocyanate (HXDI), toluene diisocyanate, hexamethylene diisocyanate (HDI), pentamethylene diisocyanate, methylene diphenyl diisocyanate, an adduct thereof, and an oligomer thereof.

17. The method according to any one of claims 1 to 16, wherein the organic phase further comprises one or more of a fragrance, a cosmetic active, a malodour counteractive, an antimicrobial agent and an insecticide.

18. The method according to any one of claims 1 to 17, wherein the organic phase further comprises a fragrance.

19. A microcapsule obtainable or obtained by a method according to any one of claims 1 to 18.

20. The microcapsule according to claim 19, having a largest dimension of 0.1 μm to 1,000 μm.

21. The microcapsule according to claim 19 or claim 20, having a shell thickness of 0.01 μm to 200 μm.

22. The microcapsule according to any one of claims 19 to 21, wherein the microcapsule is inherently biodegradable or readily biodegradable as determined by one or more tests selected from OECD TG 301B and OECD TG 301F.

23. A composition comprising a plurality of microcapsules according to any one of claims 19 to 22.

24. The composition according to claim 23, further comprising a surfactant, preservative, pH stabiliser, thickening agent, opacifier, sequestrant, propylene glycol, salt, or a combination thereof.

25. A consumer product comprising a composition according to claim 23 or claim 24, wherein the consumer product is selected from
a personal care product selected from an antiperspirant cream, a antiperspirant spray, a deodorant, a roll on, a deodorant stick, a liquid shampoo, a liquid shower gel, a liquid hair conditioner, a solid shampoo, a solid shower gel, a solid hair conditioner, a body wash, a bath fragrance, a bath bomb, a bath salt, a soap, a soap bar, a hand wash, a hand sanitiser, and hand gel, a body cream, a hand moisturiser, a face moisturiser, a hair gel, a hair wax, a hair mousse, a hair dye, a hair bleach, a shaving cream, a shaving paste, a shaving powder, a face mask, a depilatory cream, and a fake tan;
a fabric care product selected from a scent booster, a liquid laundry detergent, a liquid fabric conditioner, a detergent powder, a fabric conditioner powder, a laundry detergent sheet, a fabric detergent sheet, a fabric refresher, a laundry prewash, a laundry aid, an ironing water, and a leather care product;
a home care product selected from a furniture polish, a floor cleaner, a window cleaner, a surface cleaner, an all-purpose cleaner, a carpet shampoo, a bleach, a toilet cleaner, a toilet block, a urinal cake, a de-scaler, a disinfectant, a washing up liquid, a solid washing up soap, a dishwasher tablet, a dishwasher powder, and a dishwasher sheet;
a fragrance product selected from a diffuser, a candle, an air refresher, a reed diffuser, a incense stick, a car wash, a fine fragrance product, an Eau De Toilette, an Eau de Parfum, a cologne, a solid perfume, a perfume gel, and a scratch and sniff card; and
a textile product selected from a padded textile, and a clothing item.

26. Use of a microcapsule according to any one of claims 19 to 22 to encapsulate a cargo selected from one or more of a fragrance, a cosmetic active, a malodour counteractive, an antimicrobial agent, and an insecticide.

27. A composition for preparing a microcapsule, the composition comprising:
an aqueous phase comprising a polycation and a polyanion;
an organic phase comprising a polyisocyanate; and
a crosslinking agent,
wherein the crosslinking agent is present at an amount of up to 0.03% by weight of composition.

28. A composition for preparing a microcapsule, the composition comprising:
an aqueous phase comprising a polycation and a polyanion;
an organic phase comprising a polyisocyanate; and
a crosslinking agent,
wherein the crosslinking agent is present at an amount of up to 10 parts by weight relative to 1,000 parts by total weight of the polycation and the polyanion.

29. The composition according to claim 28, wherein the crosslinking agent is in an amount of 0.1 to 10 parts by weight relative to 1,000 parts by total weight of the polycation and the polyanion.

30. The composition according to claim 28 or claim 29, wherein the crosslinking agent is in an amount of up to 5 parts by weight relative to 1,000 parts by weight of the polycation and the polyanion.

31. The composition according to any one of claims 28 to 30, wherein the crosslinking agent is in an amount of up to 4 parts by weight relative to 1,000 parts by weight of the polycation and the polyanion.

32. The composition according to any one of claims 27 to 31, wherein the aqueous phase comprises a complex coacervate of the polycation and the polyanion.

33. The composition according to any one of claims 27 to 32, wherein the polycation is selected from a polysaccharide, a plant protein and a milk protein.

34. The composition according to any one of claims 27 to 33, wherein the polycation is a polysaccharide.

35. The composition according to any one of claims 27 to 34, wherein the polycation is chitosan.

36. The composition according to any one of claims 27 to 35, wherein the polyanion is selected from cellulose, starch, modified starch, agar, alginate, xanthan gum, plant gum, casein, zein, agarose, polyvinyl alcohol, isosorbide, derivatives thereof and salts thereof.

37. The composition according to any one of claims 27 to 36, wherein the polyanion is acacia gum.

38. The composition according to any one of claims 27 to 37, wherein the crosslinking agent is glutaraldehyde.

39. The composition according to any one of claims 27 to 38, wherein the polyisocyanate comprises an aromatic group.

40. The composition according to any one of claims 27 to 38, where the polyisocyanate comprises one or more selected from xylylene diisocyanate (XDI), hydrogenated xylylene diisocyanate (HXDI), toluene diisocyanate, hexamethylene diisocyanate (HDI), pentamethylene diisocyanate, methylene diphenyl diisocyanate, an adduct thereof, and an oligomer thereof.

41. The composition according to any one of claims 27 to 40, wherein the organic phase further comprises one or more of a fragrance, a cosmetic active, a malodour counteractive, an antimicrobial agent and an insecticide.

42. The composition according to any one of claims 27 to 41, wherein the organic phase further comprises a fragrance.
